(19) United States
(12) Patent Application Publication
(10) Pub. No.: US 2015/0112063 A1
(43) Pub. Date: Apr. 23, 2015

(54) PROCESS OF PREPARATION OF AZIMSULFURON

(71) Applicant: Rallis India Limited, Bangalore (IN)

(72) Inventors: Jivan Dhanraj Pawar, Bangalore (IN); Sanjay Sambhajirao Palimkar, Bangalore (IN); B. Sankar, Bangalaore (IN); Subhash Rajaram Kadam, Pune (IN); Rama Mohan Hindupur, Bangalore (IN); Venkatesh M. Prabhu, Bangalore (IN); Hari Narayan Pati, Bangalore (IN); Vadiraj Gopinath Suphala, Bangalore (IN); Avinash Sheshrao Mane, Bangalore (IN)

(73) Assignee: RALLIS INDIA LIMITED, Bangalore (IN)

(21) Appl. No.: 14/005,809

(22) PCT Filed: Apr. 12, 2013
(86) PCT No.: PCT/IN2013/000252

§ 371 Date: Sep. 17, 2013

(30) Foreign Application Priority Data
Jun. 26, 2012 (IN) 2537/CHE/2012

(51) Int. Cl. C07D 403/14; A01N 47/36

(52) U.S. Cl. CPC C07D 403/14; A01N 47/36
USPC 544/298

(57) ABSTRACT

The present disclosure provides process for preparation of azimsulfuron or its salts, isomers, and other derivatives thereof. The process involves treating a compound of formula I,

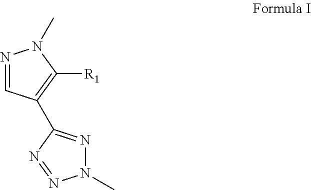

with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride; converting the sulfonyl chloride to a sulfonamide and treating the sulfonamide with a phenyl(4,6-dimethoxypyrimidin-2-yl) carbamate to obtain azimsulfuron or its salts, isomers, and other derivatives thereof.

### TECHNICAL FIELD

The present disclosure relates to process for preparation of a herbicidal sulfonamide, azimsulfuron (1-(4,6-dimethoxypyrimidin-2-yl)-3-[1-methyl-4-(2-methyl-2H-tetrazol-5-yl)pyrazol-5-ylsulfonyl]urea) or its salts, isomers, and other derivatives thereof.

### BACKGROUND

Sulfonylureas are a family of environmentally compatible herbicides that were discovered by DuPont Crop Protection in 1975 and first commercialized for wheat and barley crops in 1982. They have now been developed and commercialized worldwide in all major agronomic crops and for many specialty uses (e.g., rangeland/pasture, forestry, vegetation management). Sulfonylureas represent a major advance in global crop protection technology and have revolutionized weed control by introducing a unique mode of action. Specifically, these compounds interfere with a key enzyme required for weed cell growth acetolactate synthase. Furthermore, sulfonylureas are compatible with the global trend toward post emergence weed control and integrated pest management.

Sulfonylurea herbicides are well known in the art as being highly beneficial for controlling undesirable vegetation in agronomically desirable crops including corn, cereals such as wheat and barley. They work on a broad range of grasses and broadleaf weeds, but not on crops they are designed to protect. Crops like rice, wheat, barley, soybean, maize, and many others are able to metabolize sulfonylureas safely.

Sulfonyl urea derivatives containing pyrazole ring (pyrazole sulfonylureas) is found to exhibit pronounced herbicidal activity; especially, those in which sulfonyl urea bridge is attached to 5^{th} position of pyrazole ring (pyrazole-5-sulfonyl urea derivatives) are found to be more selective for rice plants from paddy weeds (J. pesticide Sci. 15, 531, 1990)

One type of this class of sulfonylurea herbicides, Azimsulfuron or 1-(4,6-dimethoxypyrimidin-2-yl)-3-[1-methyl-4-(2-methyl-2H-tetrazol-5-yl)pyrazol-5-ylsulfonyl]urea, is well known and has been found to be particularly useful as post emergent herbicides when used against sensitive weeds. It is used post-emergence in rice fields against a variety of annual weeds. Azimsulfuron affects sensitive weeds through inhibition of the enzyme acetolactate synthase (ALS) Inhibition of ALS leads to the cessation of cell division and subsequent growth processes in plants. Azimsulfuron is taken up mainly by leaves and shoots and, to a lesser extent, roots. Once taken up, it is translocated via both xylem and phloem. The Sulfonyl ureas are known in art and can be prepared in a number of different ways such as reaction of either sulfonamide with isocyanate, carbamate & N-carbamoyl chloride of amines or sulfonyl isocyanate & carbamate of sulfonamides with amines in presence of base respectively.

U.S. Pat. No. 4,746,353, which is incorporated herein by reference in its entirety, discloses a process of preparation of tetrazole substituted sulfonamide herbicides by reacting 3-(2-methyl-2H-tetrazole-5-yl)-1-phenyl-1H-pyrazole-5-sulfonamide with a phenyl (4,6-dimethoxypyrimidin-2-yl)carbamate. The 3-(2-methyl-2H-tetrazole-5-yl)-1-phenyl-1H-pyrazole-5-sulfonamide was reported to be prepared from 5-amino-1-phenyl-3-(2-methyl-2H-tetrazol-5-yl)-1H-pyrazole via diazotization of it and subsequent treatment of diazonium salt with sulfur dioxide and cupric chloride followed by reaction of intermediate sulfonyl chloride with aqueous ammonia.

U.S. Pat. No. 4,650,892 discloses a process of preparation of sulfonamide herbicides using aromatic sulfonyl chlorides and sulfonamides. The preparation of sulfonamide from ammonium hydroxide and sulfonyl chloride is reported in literature, e.g, Crossley et. al. J. Am. Chem. Soc. 60, 2223 (1938).

U.S. Pat. No. 4,169,719 discloses a compound of formula I and its process for preparation.

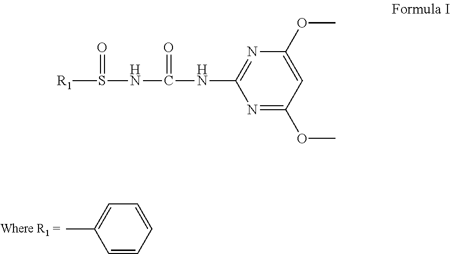

Compound of formula I was reported to be prepared by reacting substituted 2-amino pyrimidine with appropriately substituted sulfonyl isocyanate or isothiocynates.

Japanese patent JP7300403A and Chinese research article (Anhui Huagong 37(4), 10-12, 2011) disclose the process for preparation of azimsulfuron by reacting 1-methyl-4-(2-methyl-2H-tetrazol-5-yl)-1H-pyrazole-5-sulfonamide with phenyl(4,6-dimethoxypyrimidin-2-yl)carbamate in presence of 1,8-diazabicyclo[5.4.0]undec-7-ene and acetonitrile. Preparation of 1-methyl-4-(2-methyl-2H-tetrazol-5-yl)-1H-pyrazole-5-sulfonamide was reported by diazotization of 5-amino-1-methyl-4-(2-methyl-2H-tetrazole-5-yl)pyrazole followed by reaction with sulfur dioxide and cupric chloride resulting into sulfonyl chloride. The sulfonyl chloride converted into sulfonamide by treatment with aqueous ammonia in the presence of tetrahydrofuran.

The need exists, however, for newer process for preparing these herbicides, like azimsulfuron which destroy or reward weeds without causing significant damage to useful crops.

### SUMMARY

The present disclosure relates to a process for preparation of azimsulfuron or its salts, isomers, and other derivatives thereof, comprising:
treating a compound of formula I, or mixtures thereof

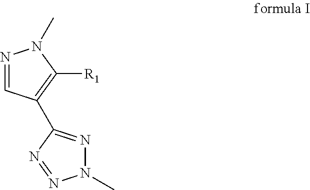

wherein,
R₁ is selected from —SH, —SCH₂Ph, —SR or —S—SR₁′; where R is a straight or a branched chain C₁₋₄ alkyl and R₁′ is a group of formula II and where * denotes the point of attachment

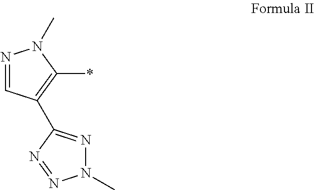 [SMILES: *c1c(-c2nnn(C)n2)cnn1C]

with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in presence of aqueous ammonia and a solvent selected from a chlorinated hydrocarbon, such as dichloromethane, 1,2-dichloroethane or chloroform; a cyclic ether such as tetrahydrofuran, or 1,4-dioxane; or a ketone such as acetone, ethyl methyl ketone or methyl isobutyl ketone; and
treating 1-methyl-4-(2-methyl-2H-tetrazol-5-yl)-1H-pyrazole-5-sulfonamide with phenyl (4,6-dimethoxypyrimidin-2-yl)carbamate in the presence of an organic base and a polar solvent to obtain azimsulfuron or its salts, isomers, and other derivatives thereof.

These and other features, aspects, and advantages of the present disclosure will become better understood with reference to the following description. This statement is provided to introduce a selection of concepts in a simplified form. This statement is not intended to identify key features or essential features of the subject matter, nor is it intended to be used to limit the scope of the subject matter.

### DETAILED DESCRIPTION

The present disclosure will be described more fully hereinafter. Indeed, the invention can be embodied in many different forms and should not be construed as limited to the embodiments set forth herein. As used in the specification, and in the appended claims, the singular forms “a”, “an”, “the”, include plural referents unless the context clearly dictates otherwise.

In the present disclosure, the following definitions will apply unless indicated explicitly.

The term “C₁₋₄ alkyl” refers to a monoradical, branched or unbranched saturated hydrocarbon chain having 1, 2, 3 or 4 carbon atoms. This term is exemplified by groups such as methyl, ethyl, n-propyl, iso-propyl, n-butyl, iso-butyl and the like.

The term “C₁-C₄ alkyl thiol” refers to an alkyl thiol group of 1 to 4 carbon atoms. The term “C₁₋₄ alkyl thiol” is exemplified by groups such as methyl thiol, ethyl thiol, n-propyl thiol, iso-propyl thiol, n-butyl thiol, iso-butyl thiol and the like.

The present disclosure provides a process for the preparation of herbicidal sulfonamide, azimsulfuron or its salts, isomers, and other derivatives thereof. Azimsulfuron is useful as an active ingredient for agrochemical, especially herbicides.

Azimsulfuron or its salts, isomers, and other derivatives thereof may be used as a foliar, or soil applied herbicides suitable for the control of many annual and perennial broadleaves species in cereals, grains, corn, sugarcane and other crops and vine control in pasture and crop land, and can also be used in pre plant or pre emergence applications.

Accordingly, the present disclosure provides a process for preparation of azimsulfuron or its salts, isomers, and other derivatives thereof, comprising:
treating a compound of formula I, or mixtures thereof,

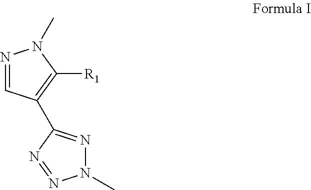

wherein,
R₁ is selected from —SH, —SCH₂Ph, —SR or —S—SR₁′; where R is a straight or a branched chain C₁₋₄ alkyl and R₁′ is a group of formula II where * denotes the point of attachment

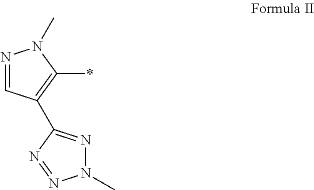 [SMILES: *c1c(-c2nnn(C)n2)cnn1C]

with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in presence of aqueous ammonia and a solvent selected from a chlorinated hydrocarbon, such as dichloromethane, 1,2-dichloroethane, or chloroform; a cyclic ether such as tetrahydrofuran, or 1,4-dioxane; or a ketone such as acetone, ethyl methyl ketone or methyl isobutyl ketone; and
treating 1-methyl-4-(2-methyl-2H-tetrazol-5-yl)-1H-pyrazole-5-sulfonamide with phenyl (4,6-dimethoxypyrimidin-2-yl)carbamate in the presence of an organic base and a polar solvent to obtain azimsulfuron or its salts, isomers, and other derivatives thereof. In accordance with the present disclosure, the oxidizing agent is suitably selected from any oxidizing agent that can facilitate formation of the sulfonyl chloride. The oxidizing agent is preferably selected from hydrogen peroxide, sodium hypochlorite, N-chlorosuccinimide or chlorine gas.

In an aspect of the present disclosure, the polar solvent is selected from N,N-dimethylformamide, N,N-dimethylacetamide, N-methyl-2-pyrrolidone, acetonitrile, acetone, dichloromethane, 1,2-dichloroethane, methanol, tetrahydrofuran, or water; and the organic base is selected from di-isopropyl ethyl amine, di-isopropyl amine, tri-n-butyl amine, triethyl amine, trimethyl amine or pyridine.

In accordance with the present disclosure, it provides a process for the preparation of azimsulfuron or its salts, isomers, and other derivatives thereof, comprising:
treating a compound of formula I, or mixtures thereof

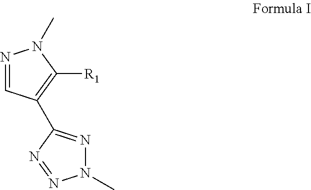

wherein, R₁ is selected from —SH, —SCH₂Ph, —SR or —S—SR₁′; where R is a straight or a branched chain C₁₋₄ alkyl and R₁′ is a group of formula II where * denotes the point of attachment

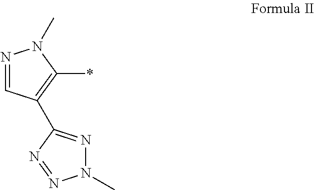 [SMILES: *c1c(-c2nnn(C)n2)cnn1C]

with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in presence of triethylamine and acetonitrile to obtain azimsulfuron or its salts, isomers, and other derivatives thereof.

In another embodiment, the compound of formula I is obtained by treating a 5-(5-halo-1-methyl-1H-pyrazole-4-yl)-2-methyl-2H-tetrazole with a thiol or an alkali hydrosulfide in presence of a solvent selected from N,N-dimethylformamide, N,N-dimethylacetamide, N-methyl-2-pyrrolidone, acetonitrile, acetone, tetrahydrofuran or water.

In an aspect of the present disclosure, the compound of formula I, wherein R₁ is selected from —SH, or —S—SR₁′; where R₁′ is a group of formula II where * denotes the point of attachment

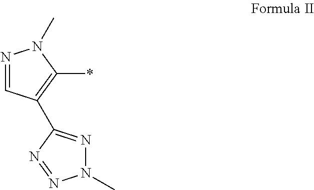 [SMILES: *c1c(-c2nnn(C)n2)cnn1C]

is obtained by treating a 5-(5-halo-1-methyl-1H-pyrazole-4-yl)-2-methyl-2H-tetrazole with an alkali metal hydrosulfide in the presence of N,N-dimethylformamide. The alkali hydrosulfide is suitably selected from sodium hydrosulfide or potassium hydrosulfide.

In another aspect of the present disclosure, the compound of formula I, wherein R₁ is selected from —SCH₂Ph or —SR, where R is a straight or a branched chain C₁₋₄ alkyl, is obtained by treating 5-(5-halo-1-methyl-1H-pyrazole-4-yl)-2-methyl-2H-tetrazole with a thiol in the presence of sodium hydride and N,N-dimethylformamide.

The thiol used in the present disclosure can be a C₁₋₄ alkyl thiol or benzyl thiol.

In yet another embodiment, the compound 5-(5-halo-1-methyl-1H-pyrazole-4-yl)-2-methyl-2H-tetrazole is obtained by halogenating 2-methyl-5-(1-methyl-1H-pyrazol-4-yl)-2H-tetrazole with a halogenating agent, selected from N-chlorosuccinimide, N-bromosuccinimide or N-iodosuccinimide in the presence of a solvent selected from N,N-dimethylformamide, N,N-dimethylacetamide, N-methyl-2-pyrrolidone, acetonitrile, acetone, dichloromethane, 1,2-dichloroethane, methanol, tetrahydrofuran, ethyl acetate, or butyl acetate.

The 5-(5-halo-1-methyl-1H-pyrazole-4-yl)-2-methyl-2H-tetrazole described in the present disclosure is obtained by treating 1-methyl-1H-pyrazole with phosphoryl chloride in the presence of N,N-dimethylformamide, to obtain 1-methyl-1H-pyrazole-4-carbaldehyde;
treating 1-methyl-1H-pyrazole-4-carbaldehyde with hydroxylamine hydrochloride in the presence of ethanol, to obtain 1-methyl-1H-pyrazole-4-carbaldehyde oxime;
treating 1-methyl-1H-pyrazole-4-carbaldehyde oxime with sodium azide in the presence of ammonium chloride and N,N-dimethylformamide, to form 5-(1-methyl-1H-pyrazole-4-yl)-2H-tetrazole;
reacting 5-(1-methyl-1H-pyrazol-4-yl)-2H-tetrazole with methyl iodide or dimethyl sulphate, in the presence of potassium hydroxide or potassium carbonate to obtain 2-methyl-5-(1-methyl-1H-pyrazol-4-yl)-2H-tetrazole or 1-methyl-5-(1-methyl-1H-pyrazol-4-yl)-1H-tetrazole or mixtures thereof; and
halogenating 2-methyl-5-(1-methyl-1H-pyrazol-4-yl)-2H-tetrazole with N-halosuccinimide, in the presence acetonitrile, to obtain a 5-(5-halo-1-methyl-1H-pyrazol-4-yl)-2-methyl-2H-tetrazole.

The regio-isomers, 2-methyl-5-(1-methyl-1H-pyrazol-4-yl)-2H-tetrazole or 1-methyl-5-(1-methyl-1H-pyrazol-4-yl)-1H-tetrazole or mixtures thereof obtained in the present disclosure were separated. The desired regio-isomer, 2-methyl-5-(1-methyl-1H-pyrazol-4-yl)-2H-tetrazole, was isolated by column chromatography. The desired regiomer, 2-methyl-5-(1-methyl-1H-pyrazol-4-yl)-2H-tetrazole is further used for the preparation of azimsulfuron, or its salts, or other derivatives thereof by the process of the present disclosure.

The 5-(5-halo-1-methyl-1H-pyrazole-4-yl)-2-methyl-2H-tetrazole described in the present disclosure can also be obtained by methylating a 5-(5-halo-1-methyl-1H-pyrazole-4-yl)-2H-tetrazole with a methylating agent, such as dimethyl sulfate, methyl iodide or methyl bromide, and a base selected from an alkali metal hydroxide base, such as sodium hydroxide, potassium hydroxide or lithium hydroxide; or an alkali metal carbonate base, such as sodium carbonate, sodium bicarbonate, potassium carbonate or cesium carbonate; or an organic base such as triethyl amine, di-isopropyl amine, di-isopropyl ethyl amine, pyridine, pyrrolidine or N,N-dimethyl aniline, in the presence of a polar solvent such as N,N-dimethylformamide, N,N-dimethylacetamide, N-methyl-2-pyrrolidone, acetonitrile, acetone, dichloromethane, 1,2-dichloroethane, methanol, tetrahydrofuran or water.

Methylation of 5-(5-halo-1-methyl-1H-pyrazole-4-yl)-2H-tetrazole as described in the present disclosure gives 5-(5-halo-1-methyl-1H-pyrazole-4-yl)-2-methyl-2H-tetrazole, or its regio-isomer 5-(5-halo-1-methyl-1H-pyrazole-4-yl)-1-methyl-1H-tetrazole, or mixtures thereof. The desired regio-isomer, 5-(5-halo-1-methyl-1H-pyrazol-4-yl)-2-methyl-2H-tetrazole), was isolated by column chromatography at this stage or at the next thiol preparation step. After this step only the desired regiomer i.e. 5-(5-halo-1-methyl-1H-pyrazole-4-yl)-2-methyl-2H-tetrazole is used for the preparation of azimsulfuron, or its salts, or other derivatives thereof.

The 5-(5-halo-1-methyl-1H-pyrazole-4-yl)-2H-tetrazole compound described in the present disclosure can be obtained by any known process in the art. Particularly, in an aspect of the present invention, this tetrazole compound used for further preparation of Azimsulfuron by the process of the present disclosure, is obtained by treating malononitrile with triethyl orthoformate in the presence of acetic anhydride to obtain 2-(ethoxymethylene)malononitrile;
treating 2-(ethoxymethylene)malononitrile with an N-methyl hydrazine sulfate or N-methyl hydrazine, and a base selected from an alkali metal hydroxide base such as sodium hydroxide, potassium hydroxide or lithium hydroxide; or an alkali metal carbonate base such as sodium carbonate, sodium bicarbonate, potassium carbonate or cesium carbonate; or an organic base such as triethyl amine, di-isopropyl amine, di-isopropyl ethyl amine, pyridine, pyrrolidine or N,N-dimethyl aniline, in the presence of an alcohol such as methanol, ethanol or propanol to obtain 5-amino-1-methyl-1H-pyrazole-4-carbonitrile;
halogenating 5-amino-1-methyl-1H-pyrazole-4-carbonitrile with sodium nitrite and a cuprous halide such as cuprous chloride, cuprous bromide or cuprous iodide, with a mineral acid such as hydrochloric acid, hydrobromic acid or sulfuric acid, to obtain 5-halo-1-methyl-1H-pyrazole-4-carbonitrile; and
treating 5-halo-1-methyl-1H-pyrazole-4-carbonitrile with sodium azide in the presence of either an ammonium halide such as ammonium chloride, ammonium bromide or ammonium iodide, and a polar solvent such as N,N-dimethylformamide, N,N-dimethylacetamide, N-methyl-2-pyrrolidone, 1,2-dichloroethane, tetrahydrofuran or water; or a tri-n-butyl tin halide such as tri-n-butyl tin chloride, tri-n-butyl tin bromide or tri-n-butyl tin iodide, and a non polar solvent such as toluene, xylene or benzene, to obtain 5-(5-halo-1-methyl-1H-pyrazole-4-yl)-2H-tetrazole).

Another embodiment of the present disclosure provides a process for preparing 5-(5-halo-1-methyl-1H-pyrazole-4-yl)-2-methyl-2H-tetrazole by a process comprising:
treating 1-methyl-1H-pyrazole with iodine and hydrogen peroxide in aqueous medium to obtain 4-iodo-1-methyl-1H-pyrazole;
treating 4-iodo-1-methyl-1H-pyrazole with potassium ferrocyanide and palladium (II) acetate in the presence of sodium carbonate, and N,N-dimethylacetamide, to obtain 1-methyl-1H-pyrazole-4-carbonitrile;
treating 1-methyl-1H-pyrazole-4-carbonitrile with sodium azide and either with tri-n-butyl tin chloride in the presence of toluene, or ammonium chloride in the presence of N,N-dimethylformamide to obtain 5-(1-methyl-1H-pyrazol-4-yl)-2H-tetrazole;
reacting 5-(1-methyl-1H-pyrazole-4-yl)-2H-tetrazole with methyl iodide and dimethyl sulphate, in the presence of potassium hydroxide and potassium carbonate to obtain 2-methyl-5-(1-methyl-1H-pyrazol-4-yl)-2H-tetrazole, or 1-methyl-5-(1-methyl-1H-pyrazol-4-yl)-1H-tetrazole and mixtures thereof; and
halogenating 2-methyl-5-(1-methyl-1H-pyrazol-4-yl)-2H-tetrazole with N-halosuccinimide, in the presence acetonitrile, to obtain 5-(5-halo-1-methyl-1H-pyrazol-4-yl)-2-methyl-2H-tetrazole.

The regio-isomers, 2-methyl-5-(1-methyl-1H-pyrazol-4-yl)-2H-tetrazole or 1-methyl-5-(1-methyl-1H-pyrazol-4-yl)-1H-tetrazole or mixtures thereof obtained in the present disclosure were separated. These regio-isomers can form azimsulfuron or its isomers further the process of the present disclosure. In a preferred embodiment, the regio-isomer, 2-methyl-5-(1-methyl-1H-pyrazol-4-yl)-2H-tetrazole, was isolated by column chromatography and was further used for the preparation of azimsulfuron, or its salts, or other derivatives thereof. In one of aspect of the present disclosure, it provides a process for preparation of azimsulfuron or its salts, isomers, and other derivatives thereof, comprising:
treating 5-(5-chloro-1-methyl-1H-pyrazole-4-yl)-2-methyl-2H-tetrazole with benzyl thiol in the presence of N,N-dimethylformamide and sodium hydride to obtain the compound 5-(5-(benzylthio)-1-methyl-1H-pyrazol-4-yl)-2-methyl-2H-tetrazole of formula I, wherein R₁ is —SCH₂Ph,

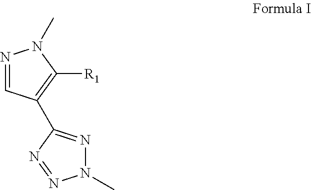

treating the compound 5-(5-(benzylthio)-1-methyl-1H-pyrazol-4-yl)-2-methyl-2H-tetrazole of formula I with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl) carbamate in the presence of triethylamine and acetonitrile to obtain azimsulfuron or its salts, isomers, and other derivatives thereof.

In another aspect of the present disclosure, it provides a process for preparation of azimsulfuron or its salts, isomers, and other derivatives thereof, comprising:
treating 5-(5-chloro-1-methyl-1H-pyrazole-4-yl)-2-methyl-2H-tetrazole with sodium hydrosulfide in the presence of N,N-dimethylformamide to obtain compounds (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof;
treating 1-methyl-4-(2-methyl-2H-tetrazol-5-yl)-1H-pyrazole-5-thiol, or its disulfide (1,2-bis-(1-methyl-4-(2-methyl-2H-tetrazol-5-yl)-1H-pyrazol-5-yl)disulfane), or mixture thereof, with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof. In accordance with the present disclosure, 1-methyl-4-(2-methyl-2H-tetrazol-5-yl)-1H-pyrazole-5-thiol is of the formula:

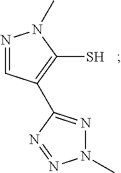

and its disulfide (1,2-bis-(1-methyl-4-(2-methyl-2H-tetrazol-5-yl)-1H-pyrazol-5-yl)disulfane), is of the formula:

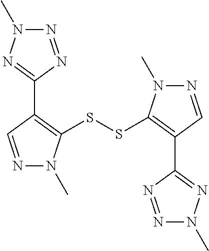

Another aspect of the present disclosure provides a process for preparation of azimsulfuron or its salts, isomers, and other derivatives thereof, comprising:
chlorinating 2-methyl-5-(1-methyl-1H-pyrazol-4-yl)-2H-tetrazole with N-chlorosuccinimide, in the presence acetonitrile, to obtain 5-(5-chloro-1-methyl-1H-pyrazol-4-yl)-2-methyl-2H-tetrazole;
treating 5-(5-chloro-1-methyl-1H-pyrazole-4-yl)-2-methyl-2H-tetrazole with sodium hydrosulfide in the presence of N,N-dimethylformamide to obtain compounds (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof;
treating 1-methyl-4-(2-methyl-2H-tetrazol-5-yl)-1H-pyrazole-5-thiol, or its disulfide 1,2-bis-(1-methyl-4-(2-methyl-2H-tetrazol-5-yl)-1H-pyrazol-5-yl)disulfane, or mixture thereof, with aqueous acetic acid and chlorine gas or sodium hypochlorite in presence of
hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof. In another embodiment, it provides a process for preparation of azimsulfuron or its salts, isomers, and other derivatives thereof, comprising:
chlorinating 2-methyl-5-(1-methyl-1H-pyrazol-4-yl)-2H-tetrazole with N-chlorosuccinimide, in the presence acetonitrile, to obtain 5-(5-chloro-1-methyl-1H-pyrazol-4-yl)-2-methyl-2H-tetrazole;
treating 5-(5-chloro-1-methyl-1H-pyrazole-4-yl)-2-methyl-2H-tetrazole with benzyl thiol in the presence of a N,N-dimethylformamide and sodium hydride to obtain compound 5-(5-(benzylthio)-1-methyl-1H-pyrazol-4-yl)-2-methyl-2H-tetrazole of formula I, wherein R₁ is —SCH₂Ph,

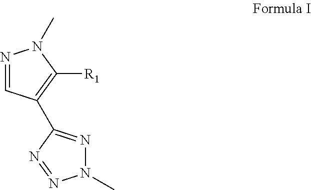

treating 5-(5-(benzylthio)-1-methyl-1H-pyrazol-4-yl)-2-methyl-2H-tetrazole with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane, to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

In yet another aspect of the present disclosure, it provides a process for preparation of azimsulfuron or its salts, isomers, and other derivatives thereof, comprising:
methylating 5-(5-chloro-1-methyl-1H-pyrazole-4-yl)-2H-tetrazole with a methylating agent, such as dimethyl sulfate, methyl iodide or methyl bromide, and potassium carbonate, in the presence of acetone to obtain 5-(5-chloro-1-methyl-1H-pyrazole-4-yl)-2-methyl-2H-tetrazole;
treating 5-(5-chloro-1-methyl-1H-pyrazole-4-yl)-2-methyl-2H-tetrazole with sodium hydrosulfide in the presence of N,N-dimethylformamide to obtain compounds 1-methyl-4-(2-methyl-2H-tetrazol-5-yl)-1H-pyrazole-5-thiol, or its disulfide (1,2-bis-(1-methyl-4-(2-methyl-2H-tetrazol-5-yl)-1H-pyrazol-5-yl)disulfane), or mixture thereof;
treating 1-methyl-4-(2-methyl-2H-tetrazol-5-yl)-1H-pyrazole-5-thiol, or its disulfide (1,2-bis-(1-methyl-4-(2-methyl-2H-tetrazol-5-yl)-1H-pyrazol-5-yl)disulfane), or mixture thereof, with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane, to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof. In another embodiment, it provides a process for preparation of azimsulfuron or its salts, isomers, and other derivatives thereof, comprising:
methylating 5-(5-chloro-1-methyl-1H-pyrazole-4-yl)-2H-tetrazole with a methylating agent, such as dimethyl sulfate, methyl iodide or methyl bromide, and potassium carbonate, in the presence of acetone to obtain 5-(5-chloro-1-methyl-1H-pyrazole-4-yl)-2-methyl-2H-tetrazole;
treating 5-(5-chloro-1-methyl-1H-pyrazole-4-yl)-2-methyl-2H-tetrazole with benzyl thiol in the presence of a N,N-dimethylformamide and sodium hydride to obtain compound 5-(5-(benzylthio)-1-methyl-1H-pyrazol-4-yl)-2-methyl-2H-tetrazole of formula I, wherein R₁ is —SCH₂Ph,

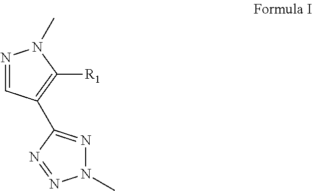

treating 5-(5-(benzylthio)-1-methyl-1H-pyrazol-4-yl)-2-methyl-2H-tetrazole with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane, to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

In another aspect of the present disclosure, it provides a process for preparation of azimsulfuron or its salts, isomers, and other derivatives thereof, comprising
treating 1-methyl-1H-pyrazole with phosphoryl chloride in the presence of N,N-dimethylformamide, to obtain 1-methyl-1H-pyrazole-4-carbaldehyde;
treating 1-methyl-1H-pyrazole-4-carbaldehyde with hydroxylamine hydrochloride in the presence of ethanol, to obtain 1-methyl-1H-pyrazole-4-carbaldehyde oxime;
treating 1-methyl-1H-pyrazole-4-carbaldehyde oxime with sodium azide in the presence of ammonium chloride and N,N-dimethylformamide, to form 5-(1-methyl-1H-pyrazole-4-yl)-2H-tetrazole;
reacting 5-(1-methyl-1H-pyrazol-4-yl)-2H-tetrazole with methyl iodide and dimethyl sulphate, in the presence of potassium hydroxide and potassium carbonate to obtain 2-methyl-5-(1-methyl-1H-pyrazol-4-yl)-2H-tetrazole;
chlorinating 2-methyl-5-(1-methyl-1H-pyrazol-4-yl)-2H-tetrazole with N-chloro succinimide, in the presence acetonitrile, to obtain 5-(5-chloro-1-methyl-1H-pyrazol-4-yl)-2-methyl-2H-tetrazole;
treating 5-(5-chloro-1-methyl-1H-pyrazole-4-yl)-2-methyl-2H-tetrazole with benzyl thiol in the presence of N,N-dimethylformamide and sodium hydride to obtain compound 5-(5-(benzylthio)-1-methyl-1H-pyrazol-4-yl)-2-methyl-2H-tetrazole of formula I, wherein R₁ is —SCH₂Ph;

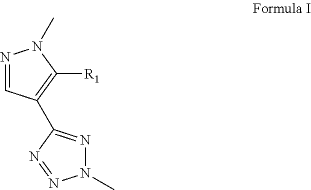

treating 5-(5-(benzylthio)-1-methyl-1H-pyrazol-4-yl)-2-methyl-2H-tetrazole aqueous acetic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane, to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof. In yet another aspect of the present disclosure, it provides a process for preparation of azimsulfuron or its salts, isomers, and other derivatives thereof, comprising
treating 1-methyl-1H-pyrazole with phosphoryl chloride in the presence of N,N-dimethylformamide, to obtain 1-methyl-1H-pyrazole-4-carbaldehyde;
treating 1-methyl-1H-pyrazole-4-carbaldehyde with hydroxylamine hydrochloride in the presence of ethanol, to obtain 1-methyl-1H-pyrazole-4-carbaldehyde oxime;
treating 1-methyl-1H-pyrazole-4-carbaldehyde oxime with sodium azide in the presence of ammonium chloride and N,N-dimethylformamide, to form 5-(1-methyl-1H-pyrazole-4-yl)-2H-tetrazole;
reacting 5-(1-methyl-1H-pyrazol-4-yl)-2H-tetrazole with methyl iodide and dimethyl sulphate, in the presence of potassium hydroxide and potassium carbonate to obtain 2-methyl-5-(1-methyl-1H-pyrazol-4-yl)-2H-tetrazole;
chlorinating 2-methyl-5-(1-methyl-1H-pyrazol-4-yl)-2H-tetrazole with N-chlorosuccinimide, in the presence acetonitrile, to obtain 5-(5-chloro-1-methyl-1H-pyrazol-4-yl)-2-methyl-2H-tetrazole;
treating 5-(5-chloro-1-methyl-1H-pyrazole-4-yl)-2-methyl-2H-tetrazole with sodium hydrosulfide in the presence of N,N-dimethylformamide to obtain the compounds (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof;
treating 1-methyl-4-(2-methyl-2H-tetrazol-5-yl)-1H-pyrazole-5-thiol, or its disulfide (1,2-bis-(1-methyl-4-(2-methyl-2H-tetrazol-5-yl)-1H-pyrazol-5-yl)disulfane), or mixture thereof, with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane, to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

In another aspect of the present disclosure, it provides a process for preparation of azimsulfuron or its salts, isomers, and other derivatives thereof, comprising:
treating malononitrile with triethyl orthoformate in the presence of acetic anhydride to obtain 2-(ethoxymethylene) malononitrile;
treating 2-(ethoxymethylene) malononitrile with N-methyl hydrazine sulfate or N-methyl hydrazine and triethyl amine, in the presence of methanol to obtain 5-amino-1-methyl-1H-pyrazole-4-carbonitrile;
chlorinating 5-amino-1-methyl-1H-pyrazole-4-carbonitrile with sodium nitrite and cuprous chloride, with hydrochloric acid to obtain 5-chloro-1-methyl-1H-pyrazole-4-carbonitrile;
treating 5-chloro-1-methyl-1H-pyrazole-4-carbonitrile with sodium azide in the presence of either ammonium chloride, and N,N-dimethylformamide, or tri-n-butyl tin chloride and toluene, to obtain 5-(5-chloro-1-methyl-1H-pyrazole-4-yl)-2H-tetrazole);
methylating 5-(5-chloro-1-methyl-1H-pyrazole-4-yl)-2H-tetrazole with a methylating agent, such as dimethyl sulfate, methyl iodide or methyl bromide; and potassium carbonate, in the presence of acetone to obtain 5-(5-chloro-1-methyl-1H-pyrazole-4-yl)-2-methyl-2H-tetrazole;
treating 5-(5-chloro-1-methyl-1H-pyrazole-4-yl)-2-methyl-2H-tetrazole with benzyl thiol in the presence of N,N-dimethylformamide and sodium hydride to obtain compound 5-(5-(benzylthio)-1-methyl-1H-pyrazol-4-yl)-2-methyl-2H-tetrazole of formula I, wherein R₁ is —SCH₂Ph;

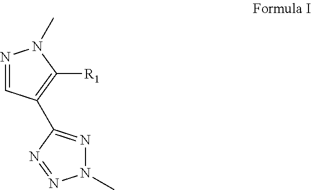

treating 5-(5-(benzylthio)-1-methyl-1H-pyrazol-4-yl)-2-methyl-2H-tetrazole with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane, to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

Another embodiment provides a process for preparation of azimsulfuron or its salts, isomers, and other derivatives thereof, comprising:
treating malononitrile with triethyl orthoformate in the presence of acetic anhydride to obtain 2-(ethoxymethylene) malononitrile;
treating 2-(ethoxymethylene) malononitrile with N-methyl hydrazine sulfate and triethyl amine, in the presence of methanol to obtain 5-amino-1-methyl-1H-pyrazole-4-carbonitrile;
chlorinating 5-amino-1-methyl-1H-pyrazole-4-carbonitrile with sodium nitrite and cuprous chloride, with hydrochloric acid to obtain 5-chloro-1-methyl-1H-pyrazole-4-carbonitrile;
treating 5-chloro-1-methyl-1H-pyrazole-4-carbonitrile with sodium azide in the presence of either ammonium chloride, and N,N-dimethylformamide, or tri-n-butyl tin chloride and toluene, to obtain 5-(5-chloro-1-methyl-1H-pyrazole-4-yl)-2H-tetrazole);
methylating 5-(5-chloro-1-methyl-1H-pyrazole-4-yl)-2H-tetrazole with dimethyl sulfate and potassium carbonate, in the presence of acetone to obtain 5-(5-chloro-1-methyl-1H-pyrazole-4-yl)-2-methyl-2H-tetrazole;
treating 5-(5-chloro-1-methyl-1H-pyrazole-4-yl)-2-methyl-2H-tetrazole with sodium hydrosulfide in the presence of a N,N-dimethylformamide to obtain the compounds (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof;
treating 1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof, with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane, to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

Still another embodiment of present disclosure provides a process for preparation of azimsulfuron or its salts, isomers, and other derivatives thereof, comprising:
treating 1-methyl-1H-pyrazole with iodine and hydrogen peroxide in aqueous medium to obtain 4-iodo-1-methyl-1H-pyrazole;
treating 4-iodo-1-methyl-1H-pyrazole with potassium ferrocyanide and palladium (II) acetate in the presence of sodium carbonate, and N,N-dimethylacetamide, to obtain 1-methyl-1H-pyrazole-4-carbonitrile;
treating 1-methyl-1H-pyrazole-4-carbonitrile with sodium azide and either with tri-n-butyl tin chloride in the presence of toluene, or ammonium chloride in the presence of N,N-dimethylformamide to obtain 5-(1-methyl-1H-pyrazol-4-yl)-2H-tetrazole;
reacting 5-(1-methyl-1H-pyrazole-4-yl)-2H-tetrazole with methyl iodide and dimethyl sulphate, in the presence of potassium hydroxide and potassium carbonate to obtain 2-methyl-5-(1-methyl-1H-pyrazol-4-yl)-2H-tetrazole;
chlorinating 2-methyl-5-(1-methyl-1H-pyrazol-4-yl)-2H-tetrazole with N-chlorosuccinimide, in the presence acetonitrile, to obtain 5-(5-chloro-1-methyl-1H-pyrazol-4-yl)-2-methyl-2H-tetrazole.
treating 5-(5-chloro-1-methyl-1H-pyrazole-4-yl)-2-methyl-2H-tetrazole with benzyl thiol in the presence of N,N-dimethylformamide and sodium hydride to obtain compound 5-(5-(benzylthio)-1-methyl-1H-pyrazol-4-yl)-2-methyl-2H-tetrazole of formula I, wherein R₁ is —SCH₂Ph,

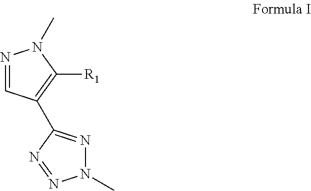

treating 5-(5-(benzylthio)-1-methyl-1H-pyrazol-4-yl)-2-methyl-2H-tetrazole with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane, to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

Yet another embodiment of present disclosure provides a process for preparation of azimsulfuron or its salts, isomers, and other derivatives thereof, comprising:
treating 1-methyl-1H-pyrazole with iodine and hydrogen peroxide in aqueous medium to obtain 4-iodo-1-methyl-1H-pyrazole;
treating 4-iodo-1-methyl-1H-pyrazole with potassium ferrocyanide and palladium (II) acetate in the presence of sodium carbonate, and N,N-dimethylacetamide, to obtain 1-methyl-1H-pyrazole-4-carbonitrile;
treating 1-methyl-1H-pyrazole-4-carbonitrile with sodium azide and either with tri-n-butyl tin chloride in the presence of toluene, or ammonium chloride in the presence of N,N-dimethylformamide to obtain 5-(1-methyl-1H-pyrazol-4-yl)-2H-tetrazole;
reacting 5-(1-methyl-1H-pyrazole-4-yl)-2H-tetrazole with methyl iodide or dimethyl sulphate, in the presence of potassium hydroxide or potassium carbonate to obtain 2-methyl-5-(1-methyl-1H-pyrazol-4-yl)-2H-tetrazole;
chlorinating 2-methyl-5-(1-methyl-1H-pyrazol-4-yl)-2H-tetrazole with N-chlorosuccinimide, in the presence acetonitrile, to obtain 5-(5-chloro-1-methyl-1H-pyrazol-4-yl)-2-methyl-2H-tetrazole;
treating 5-(5-chloro-1-methyl-1H-pyrazole-4-yl)-2-methyl-2H-tetrazole with sodium hydrosulfide in the presence of a N,N-dimethylformamide to obtain compounds (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof;
treating 1-methyl-4-(2-methyl-2H-tetrazol-5-yl)-1H-pyrazole-5-thiol, or its disulfide (1,2-bis-(1-methyl-4-(2-methyl-2H-tetrazol-5-yl)-1H-pyrazol-5-yl)disulfane), or mixture thereof, with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane, to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

The compound azimsulfuron or its salts, isomers, and other derivatives thereof can be prepared as described above in detail; is outlined in the reaction schemes as described hereinafter, and the description thereof.

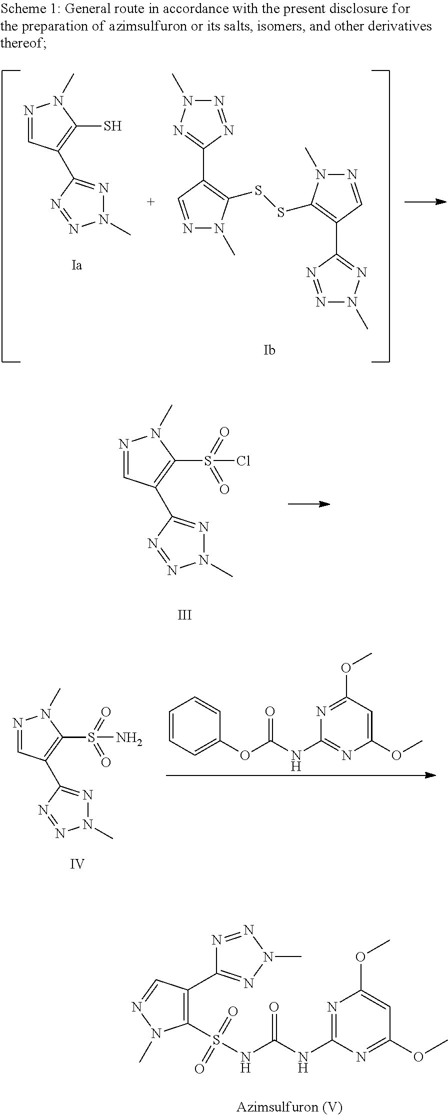

1-methyl-4-(2-methyl-2H-tetrazol-5-yl)-1H-pyrazole-5-sulfonyl chloride (III) is prepared from reaction of the compound (1-methyl-4-(2-methyl-2H-tetrazol-5-yl)-1H-pyrazole-5-thiol) (Ia), or its disulfide (1,2-bis-(1-methyl-4-(2-methyl-2H-tetrazol-5-yl)-1H-pyrazol-5-yl)disulfane) (Ib), or mixture thereof, with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane Compound (III) is converted to 1-methyl-4-(2-methyl-2H-tetrazol-5-yl)-1H-pyrazole-5-sulfonamide (IV) by treatment with aqueous ammonia and 1,2-dichloroethane; and by reacting compound(IV) with phenyl (4,6-dimethoxypyrimidin-2-yl) carbamate in the presence of triethylamine and acetonitrile, the compound azimsulfuron (V) is obtained.

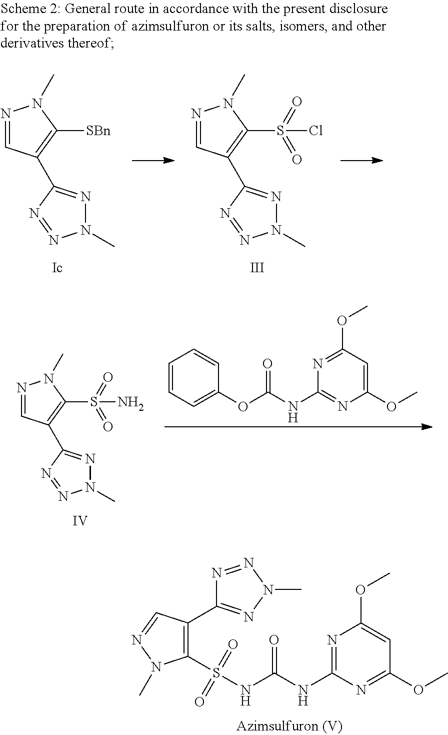

Compound (III) is prepared from reaction of the compound 5-(5-(benzylthio)-1-methyl-1H-pyrazol-4-yl)-2-methyl-2H-tetrazole of formula (Ic) with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane. Compound (III) is converted to compound (IV) by treatment with aqueous ammonia and 1,2-dichloroethane; and further to Azimsulfuron by treating compound (IV) with phenyl(4,6-dimethoxypyrimidin-2-yl) carbamate in the presence of triethylamine and acetonitrile, as described in Scheme 1.

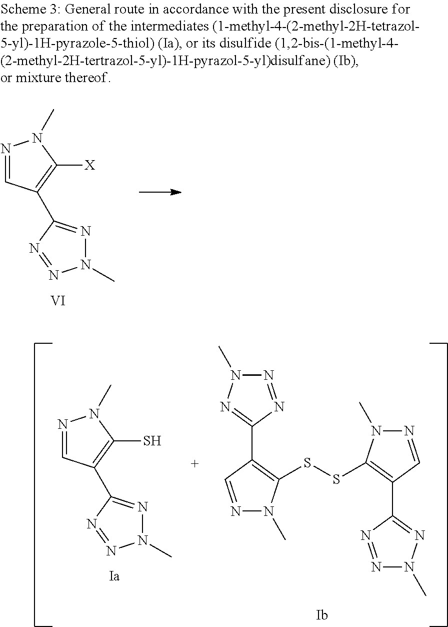

Compound 5-(5-halo-1-methyl-1H-pyrazole-4-yl)-2-methyl-2H-tetrazole (VI) is treated with sodium hydrosulfide in the presence of N,N-dimethylformamide to obtain the compound (1-methyl-4-(2-methyl-2H-tetrazol-5-yl)-1H-pyrazole-5-thiol) (Ia), or its disulfide (1,2-bis-(1-methyl-4-(2-methyl-2H-tetrazol-5-yl)-1H-pyrazol-5-yl)disulfane) (Ib), or mixture thereof. The reaction forms product obtained is a mixture of the desired thiol and its disulfide. In accordance with the present disclosure, the thiol or the disulfide may be further used as a mixture or after separating from the mixture.

Compound (VI) can be prepared by methylating 5-(5-halo-1-methyl-1H-pyrazol-4-yl)-2H-tetrazole (VII) with reagents such as dimethyl sulfate, methyl iodide or methyl bromide and potassium carbonate, in the presence of acetone.

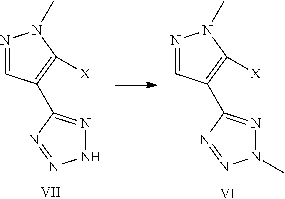

In the compound of formula VI or VII above, X is selected from Cl, Br, or I.

Compound (VI) can be also prepared by halogenating 2-methyl-5-(1-methyl-1H-pyrazol-4-yl)-2H-tetrazole (XIII) with reagents such as N-chlorosuccinimide, N-bromosuccinimide, or N-iodosuccinimide in the presence of acetonitrile.

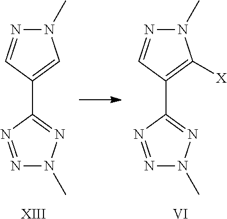

In the compound of formula VI above, X is selected from Cl, Br, or I.

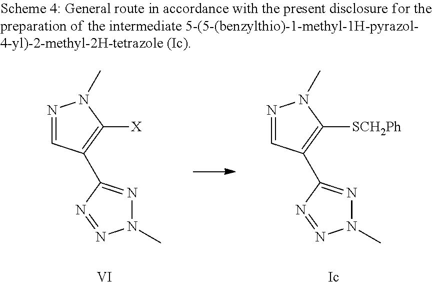

Compound (VI) treated with benzyl thiol in the presence of N,N-dimethylformamide and sodium hydride yields 5-(5-(benzylthio)-1-methyl-1H-pyrazol-4-yl)-2-methyl-2H-tetrazole (Ic).

In the compound of formula VI above, X is selected from Cl, Br, or I

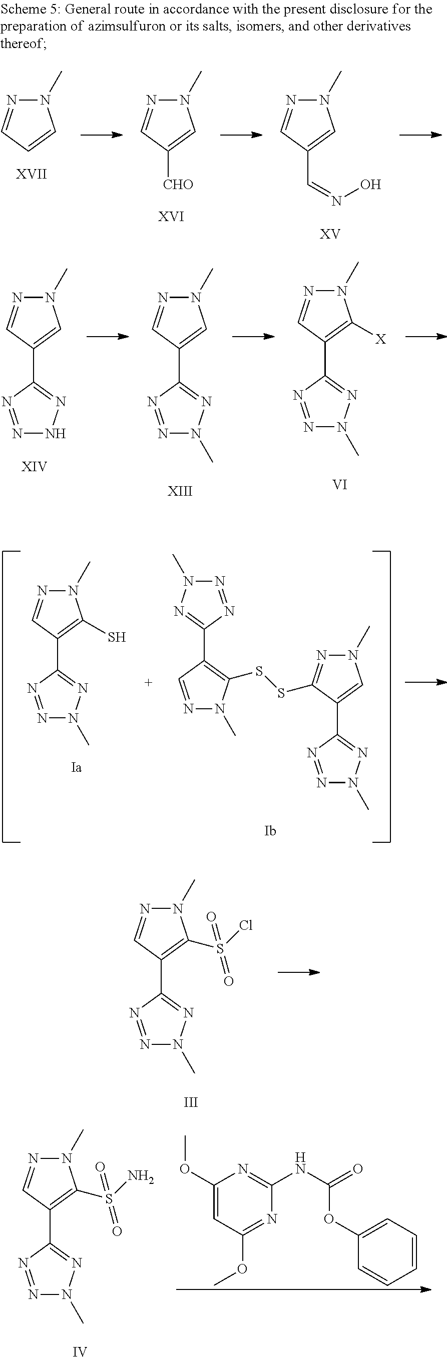

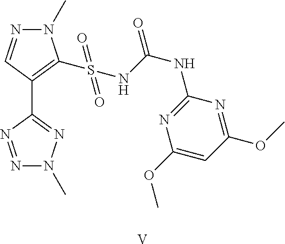

1-methyl-1H-pyrazole (XVII) is reacted with phosphoryl chloride in the presence of N,N-dimethylformamide to yield 1-methyl-1H-pyrazole-4-carbaldehyde (XVI), treating of 1-methyl-1H-pyrazole-4-carbaldehyde (XVI) with hydroxyl amine hydrochloride in presence of ethanol or ethyl acetate to obtain 1-methyl-1H-pyrazole-4-carbaldehyde oxime (XV).

The compound 1-methyl-1H-pyrazole-4-carbaldehyde oxime (XV) is subjected to a cyclization reaction in the presence of sodium azide and N,N-dimethylformamide by heating at 50-120° C., preferably 90-100° C. to obtain 5-(1-methyl-1H-pyrazol-4-yl)-2H-tetrazole (XIV).

Compound (XIV) is further converted to 2-methyl-5-(1-methyl-1H-pyrazol-4-yl)-2H-tetrazole (XIII) by treating with a methylating reagents such as dimethyl sulfate, methyl iodide or methyl bromide and potassium carbonate, in the presence of acetone.

Halogenating the compound of formula (XIII) with a halogenating agent such as N-chlorosuccinimide, N-bromosuccinimide or N-iodosuccinimide in presence of acetonitrile, yields 5-(5-halo-1-methyl-1H-pyrazole-4-yl)-2H-tetrazole) (VI).

Treating compound (VI) with sodium hydrosulfide in the presence of N,N-dimethylformamide yields the compound (1-methyl-4-(2-methyl-2H-tetrazol-5-yl)-1H-pyrazole-5-thiol) (Ia), or its disulfide (1,2-bis-(1-methyl-4-(2-methyl-2H-tetrazol-5-yl)-1H-pyrazol-5-yl)disulfane) (Ib), or mixture thereof. The thiol compound (Ia), its disulfide (Ib) or the mixtures thereof when treated with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane, yields 1-methyl-4-(2-methyl-2H-tetrazol-5-yl)-1H-pyrazole-5-sulfonyl chloride (III).

The compound (III) thus obtained is converted to 1-methyl-4-(2-methyl-2H-tetrazol-5-yl)-1H-pyrazole-5-sulfonamide (IV) by treatment with aqueous ammonia and 1,2-dichloroethane.

Reacting compound (IV) with phenyl (4,6-dimethoxypyrimidin-2-yl) carbamate in the presence of triethylamine and acetonitrile results azimsulfuron (V).

In the compound of formula VI, X is selected from Cl, Br, or I.

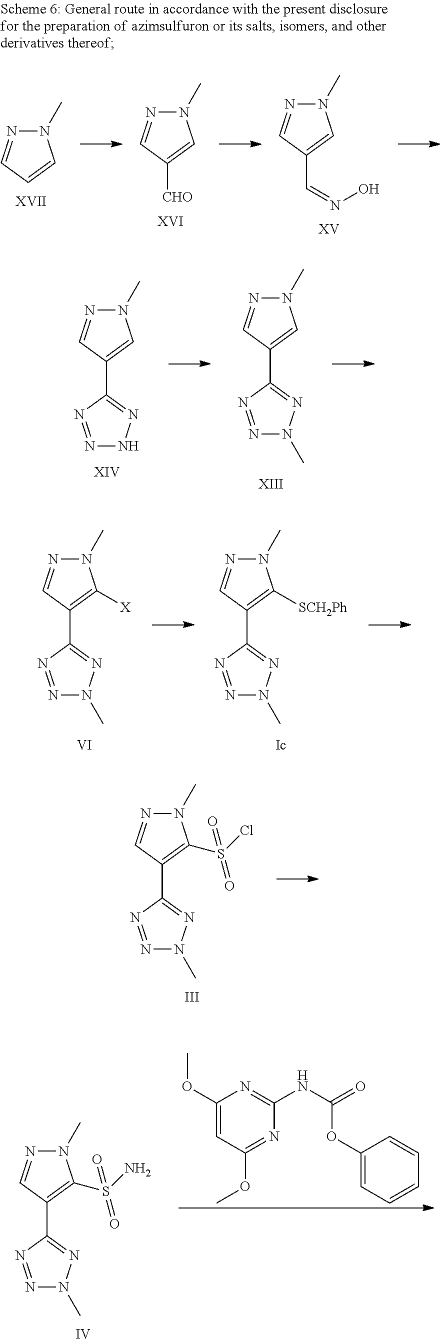

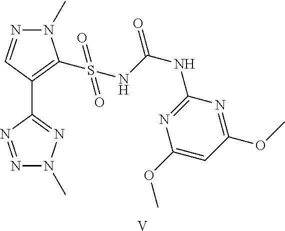

In the reaction scheme 5, compound 5-(5-halo-1-methyl-1H-pyrazole-4-yl)-2-methyl-2H-tetrazole (VI) can also form compound 5-(5-(benzylthio)-1-methyl-1H-pyrazol-4-yl)-2-methyl-2H-tetrazole (Ic), as shown in the scheme 6 above by treating compound (VI) with benzyl thiol in the presence of N,N-dimethylformamide and sodium hydride.

Compound (III) or 1-methyl-4-(2-methyl-2H-tetrazol-5-yl)-1H-pyrazole-5-sulfonyl chloride can be prepared from compound (Ic) by treating it with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane.

The process as described in Scheme 5 can be followed for obtaining Azimsulfuron from compound III thus formed by scheme 6.

In the compound of formula VI, X is selected from Cl, Br, or I.

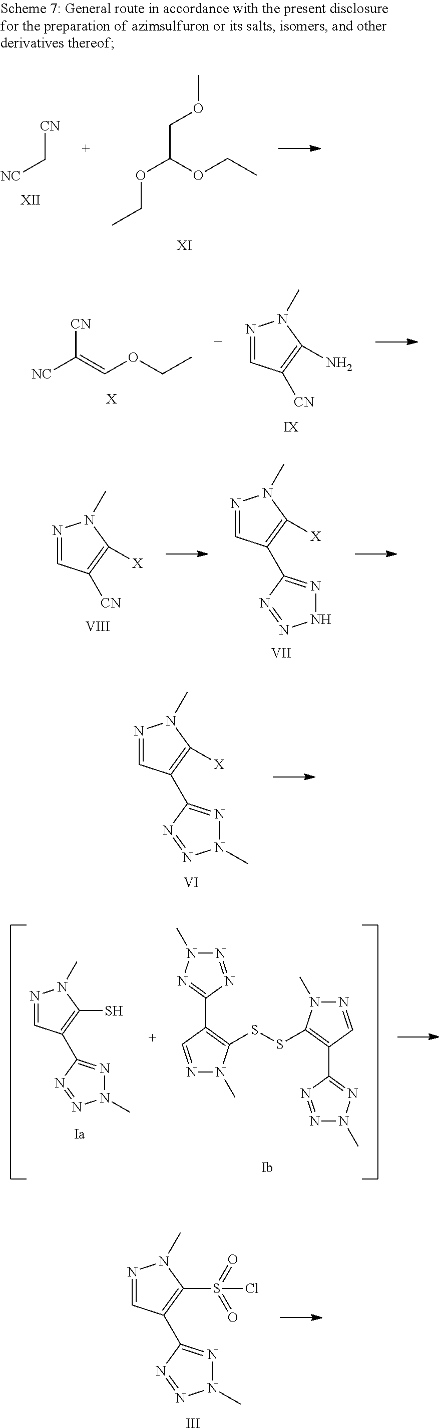

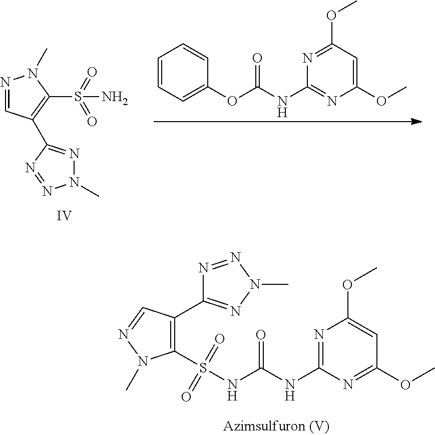

In the compound of formula VI, VII or VIII above, X is selected from Cl, Br, or I.

Malononitrile (XII) is reacted with triethyl orthoformate (XI) to yield 2-(ethoxymethylene) malononitrile (X), using acetic anhydride at a temperature in the range 105-110° C. for 2 to 3 hours.

The compound 2-(ethoxymethylene)malononitrile (X) is subjected to a cyclization reaction to obtain 5-amino-1-methyl-1H-pyrazole-4-carbonitrile (IX) in the presence of N-methyl hydrazine sulfate, N-methyl hydrazine or triethyl amine and methanol, for about 1-2 hours at reflux temperature.

Compound (IX) is further converted to 5-halo-1-methyl-1H-pyrazole-4-carbonitrile (VIII) by treatment with a halogenating agent such as a cuprous halide selected from cuprous chloride, cuprous bromide or cuprous iodide, and sodium nitrite in the presence of hydrochloric acid, hydrobromic acid, or sulfuric acid at room temperature. This compound (VIII) when reacted with an optionally substituted sodium azide in presence of either ammonium halide selected from ammonium chloride, ammonium bromide or ammonium iodide and N,N-dimethylformamide, or a tri-n-butyl tin halide such as tri-n-butyl tin chloride, tri-n-butyl tin bromide or tri-n-butyl tin iodide, and toluene at a temperature range from 50 to 100° C. preferably at 100° C. to 120° C. yields the compound 5-(5-halo-1-methyl-1H-pyrazole-4-yl)-2H-tetrazole) (VII).

Compound (VII) is further converted to 5-(5-halo-1-methyl-1H-pyrazole-4-yl)-2-methyl-2H-tetrazole (VI) by treatment with a methylating such as dimethyl sulfate, methyl iodide or methyl bromide and potassium carbonate, in the presence of acetone. Treating compound (VI) with sodium hydrosulfide in the presence of N,N-dimethylformamide yields the compound (1-methyl-4-(2-methyl-2H-tetrazol-5-yl)-1H-pyrazole-5-thiol) (Ia), or its disulfide (1,2-bis-(1-methyl-4-(2-methyl-2H-tetrazol-5-yl)-1H-pyrazol-5-yl)disulfane) (Ib), or mixture thereof. The thiol compound (Ia), its disulfide (Ib) or the mixtures thereof when treated with aqueous acetic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane, yields 1-methyl-4-(2-methyl-2H-tetrazol-5-yl)-1H-pyrazole-5-sulfonyl chloride (III).

The compound (III) thus obtained is converted to 1-methyl-4-(2-methyl-2H-tetrazol-5-yl)-1H-pyrazole-5-sulfonamide (IV) by treatment with aqueous ammonia and 1,2-dichloroethane. Reacting compound (IV) with phenyl (4,6-dimethoxypyrimidin-2-yl)carbamate in the presence of triethylamine and acetonitrile yields azimsulfuron (V).

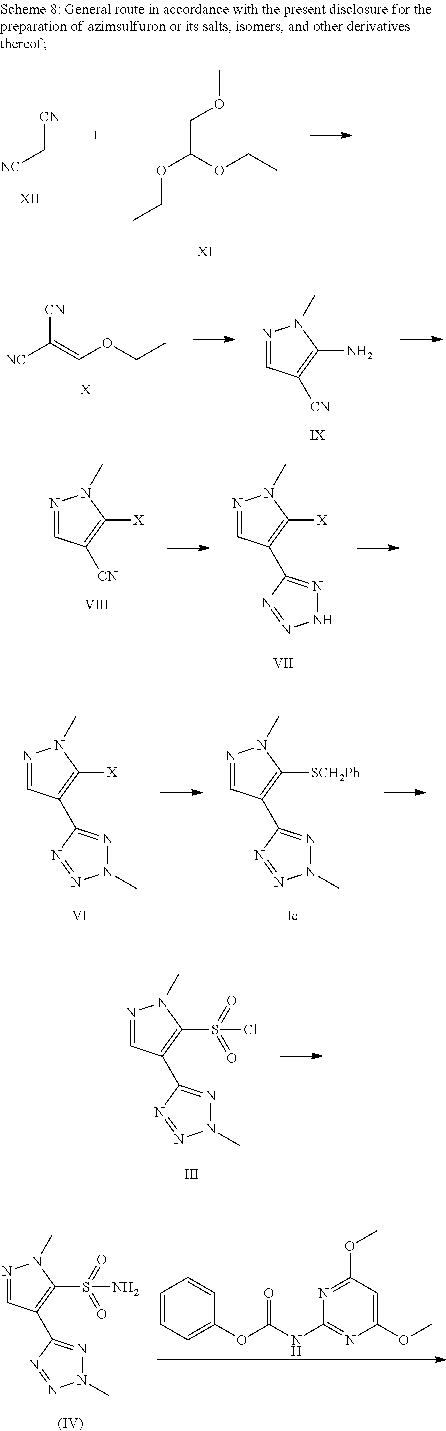

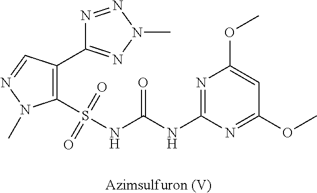 [SMILES: COc1cc(OC)nc(NC(=O)NS(=O)(=O)c2c(-c3nnn(C)n3)cnn2C)n1 ; "Azimsulfuron (V)"]

In the reaction scheme 7, compound 5-(5-halo-1-methyl-1H-pyrazole-4-yl)-2-methyl-2H-tetrazole (VI) can also form compound 5-(5-(benzylthio)-1-methyl-1H-pyrazol-4-yl)-2-methyl-2H-tetrazole (Ic), as shown in the scheme 6 above by treating compound (VI) with benzyl thiol in the presence of N,N-dimethylformamide and sodium hydride.

Compound (III) or 1-methyl-4-(2-methyl-2H-tetrazol-5-yl)-1H-pyrazole-5-sulfonyl chloride can be prepared from compound (Ic) by treating it with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane.

The process as described in Scheme 7 can be followed for obtaining Azimsulfuron from compound III thus formed by scheme 8.

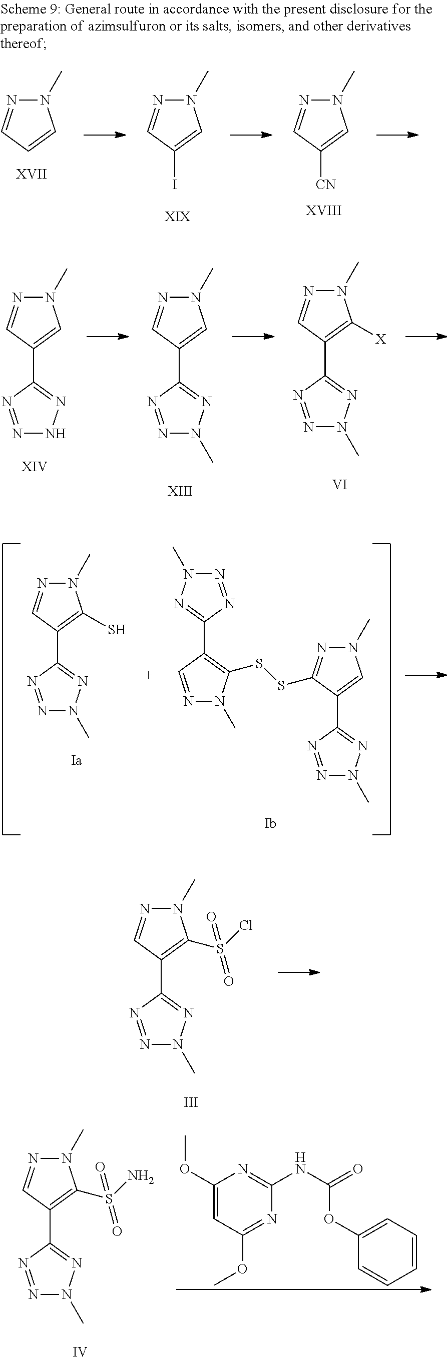

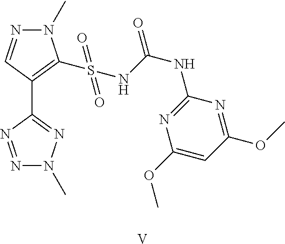

1-methyl-1H-pyrazole (XVII) is reacted with iodine in the presence of oxidizing agent in aqueous media to obtain 4-iodo-1-methyl-1H-pyrazole (XIX),

Treating of 4-iodo-1-methyl-1H-pyrazole (XIX) with sodium ferrocyanide in presence of palladium(II) acetate, sodium carbonate and N,N-dimethylacetamide to obtain 1-methyl-1H-pyrazole-4-carbonitrile (XVIII).

The compound 1-methyl-1H-pyrazole-4-carbonitrile (XVIII) is converted to 5-(1-methyl-1H-pyrazol-4-yl)-2H-tetrazole (XIV) by treatment with sodium azide with either tri-n-butyl tin chloride in the presence of toluene or ammonium chloride in presence of dimethylformamide by heating at 100-110° C. about 10-12 hours.

The process as described in Scheme 5 can be followed for obtaining Azimsulfuron from compound (X) thus formed by scheme 9.

In the compound of formula VI, X is selected from Cl, Br, or I.

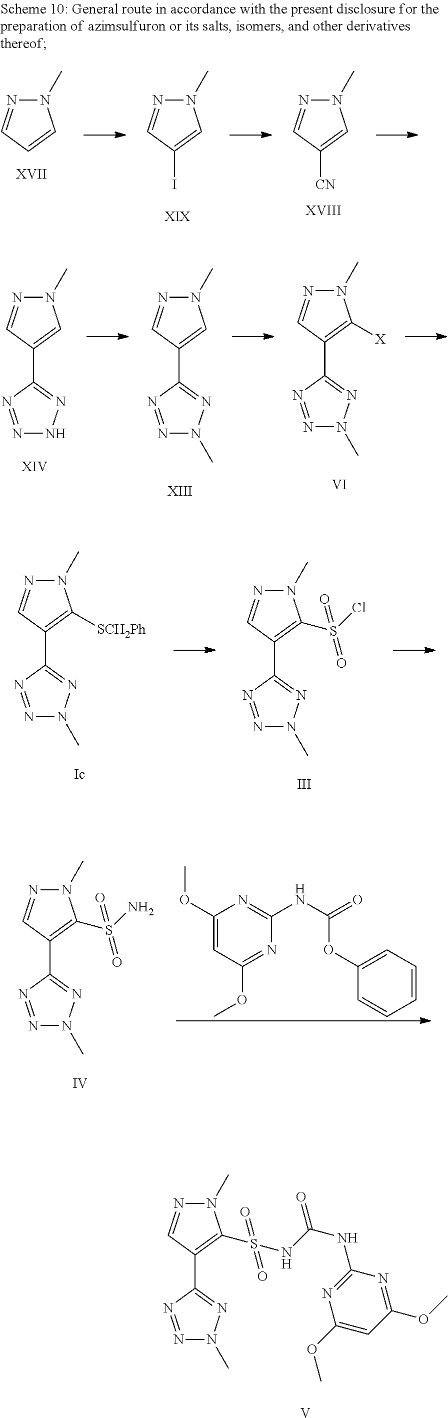

In the reaction scheme 10, compound 5-(5-halo-1-methyl-1H-pyrazole-4-yl)-2-methyl-2H-tetrazole (VI) can also form compound 5-(5-(benzylthio)-1-methyl-1H-pyrazol-4-yl)-2-methyl-2H-tetrazole (Ic), as shown in the scheme 9 above by treating compound (VI) with benzyl thiol in the presence of N,N-dimethylformamide and sodium hydride.

Compound (III) or 1-methyl-4-(2-methyl-2H-tetrazol-5-yl)-1H-pyrazole-5-sulfonyl chloride can be prepared from compound (Ic) reacted with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane.

The process as described in Scheme 5 can be followed for obtaining Azimsulfuron from compound III thus formed by scheme 10.

In the compound of formula VI, X is selected from Cl, Br, or I.

### ABBREVIATIONS

The following abbreviations are employed in the examples and elsewhere herein:

### DMF: N,N-dimethylformamide,

### DCE: Dichloroethane,

### THF: Tetrahydrofuran,

DMS: Dimethyl sulfate,
AlCl₃: Aluminium chloride,
AlBr₃: Aluminium bromide,
AlI₃: Aluminium iodide,
H₂O₂: Hydrogen peroxide,
Ac₂O: Acetic anhydride,

### DCM: Dichloromethane,

HCl: Hydrochloric acid,
IPA: Isopropyl alcohol,
DI water: De-ionized water,
DM water: Demineralised water,
M/C: Moisture content,
MP: Melting point,
BP: Boiling point,
w: weight,
w/w: weight/weight.

### EXAMPLES

The disclosure will now be illustrated with working examples, which is intended to illustrate the working of disclosure and not intended to take restrictively to imply any limitations on the scope of the present disclosure.

### Example 1

### Preparation of Azimsulfuron

### Step 1: 1-methyl-1H-pyrazole-4-carbaldehyde

A mixture of phosphoryl chloride (467 gm, 3.045 moles) and DMF was stirred for 1 hour at 0-5° C. The reaction is exothermic so temperature should be below 5° C. To this slowly added 1-methyl-1H-pyrazole (50 gm, 0.6090 moles) and stirred for 30 min at 0-5° C. Then slowly raised the temperature from 20-30° C. to 60-70° C. and stirred for 10-15 hours. Progress of the reaction was monitored by HPLC. The reaction mixture was cooled to 0-5° C., to this added ethyl acetate (250 ml), DM water (1 L) and aqueous ammonia solution (350 ml, pH-6 to 7). The reaction mixture was stirred for 1 hour at 20-30° C. The inorganic salt obtained was filtered off, washed with ethyl acetate (250 ml). The filtrate was transferred in to a separating funnel. The reaction mixture was allowed to settle. The organic layer was separated and aqueous layer was extracted with ethyl acetate (250 ml). The entire organic layer was transferred to the reaction vessel, stirred for 10-15 min and separated the final aqueous layer and organic layer. The combined organic layer having 1-methyl-1H-pyrazole-4-carbaldehyde and ethyl acetate was used as such for Step 2 without any purification or isolation at this step.

### Step 2: 1-methyl-1H-pyrazole-4-carbaldehyde oxime

A mixture of the combined organic layer having 1-methyl-1H-pyrazole-4-carbaldehyde and ethyl acetate obtained from step 1, hydroxylamine hydrochloride (63.48 gm, 0.9135 moles) were refluxed at 70-80° C. for 2-4 hours. The progress of the reaction was monitored by HPLC. The reaction mixture was cooled to 0-5° C., to this added DM water (100 ml) and 25% aqueous ammonia solution (100 ml) at 0-5° C. The reaction mixture was stirred for 10 min at 20-30° C. The organic layer was separated and aqueous layer was extracted with ethyl acetate (250 ml). The entire organic layer was transferred to the reaction vessel and washed with 10% brine solution (500 ml). Stirred for 10 min and separated the final aqueous layer and organic layer. The organic layer was evaporated to obtain the 1-methyl-1H-pyrazole-4-carbaldehyde oxime.

| Dry wt | 60 gm |
|---|---|
| Yield | 1.2 w/w (79%) |
| HPLC purity | 99.78% |

### Step 3: 5-(1-methyl-1H-pyrazol-4-yl)-2H-tetrazole

1-methyl-1H-pyrazole-4-carbaldehyde oxime (84 gm, 0.6713 moles) obtained in step 2, ammonium chloride (89.77 gm, 1.6783 moles), sodium azide (109.11 gm, 1.6783 moles) were taken in DMF (420 ml). The reaction mixture was stirred for 10-12 hours at 110-120° C. The progress of the reaction was monitored by HPLC. To the above reaction mixture at 0-10° C., added DM water (500 ml). Acidify reaction mixture using dil. HCl (5N). The reaction mixture was stirred for 2 hours at 0-10° C., filtered the product, washed with cold DM water (200 ml) and dried the product, 5-(1-methyl-1H-pyrazol-4-yl)-2H-tetrazole at 60-70° C.

| Dry wt | 75 gm |
|---|---|
| Yield | 0.89 w/w (74%) |
| HPLC purity | 99.98% |

### Step 4: 2-methyl-5-(1-methyl-1H-pyrazol-4-yl)-2H-tetrazole

A mixture of 5-(1-methyl-1H-pyrazol-4-yl)-2H-tetrazole (100 gm, 0.6660 moles), potassium hydroxide (74.74 gm, 1.332 moles) and acetonitrile (500 ml) were stirred for 10 min at 20-25° C. To the above mixture added methyl iodide (378.12 gm, 2.664 moles), stirred for 10-15 hours at 20-25° C. and progress of the reaction was monitored by HPLC. Excess acetonitrile was recovered at 60-70° C. at reduced pressure. DCM (500 ml) and DM water (500 ml) was added to the reaction mixture, stirred for 30 min and the reaction mixture was kept undisturbed for 15 min. The organic layer was separated and aqueous layer was extracted with DCM (500 ml), stirred for 10 min and separated the final aqueous layer and organic layer. The organic layer was evaporated to obtain 2-methyl-5-(1-methyl-1H-pyrazol-4-yl)-2H-tetrazole, or 1-methyl-5-(1-methyl-1H-pyrazol-4-yl)-2H-tetrazole, or mixture thereof. The desired regio-isomer, 2-methyl-5-(1-methyl-1H-pyrazol-4-yl)-2H-tetrazole, was isolated by column chromatography using ethyl acetate and hexane as eluent.

| Dry wt | 46 gm |
|---|---|
| Yield | 0.46 w/w (42%) |
| HPLC purity | 99.97 |

### Step 5: 5-(5-chloro-1-methyl-1H-pyrazol-4-yl)-2-methyl-2H-tetrazole

To a solution of 2-methyl-5-(1-methyl-1H-pyrazol-4-yl)-2H-tetrazole (10 gm, 0.609 moles) in acetonitrile (50 ml) was added N-chlorosuccinimide (8.22 gm, 0.0615 moles). The reaction mixture was stirred for 30 min at 60° C. Progress of the reaction was monitored by HPLC. Excess acetonitrile was recovered at 40-50° C. under reduced pressure. DI water (100 ml) was poured to the crude product at 20-25° C. The reaction mixture was stirred for 1 hours at 20-25° C., filtered the product, washed with cold DM water (50 ml) and dried the product, 5-(5-chloro-1-methyl-1H-pyrazol-4-yl)-2-methyl-2H-tetrazole at 60-70° C.

| | |
|---|---|
| Dry wt | 12 gm |
| Yield | 1.2 w/w (92%) |
| HPLC purity | 99.51% |
| MP | 124-127° C. |

### Step 6: 5-(5-(benzylsulfanyl)-1-methyl-1H-pyrazol-4-yl)-2-methyl-2H-tetrazole

To the above product (10 gm, 0.0505 moles) obtained from step 5, DMF (30 ml), sodium hydride (60% in oil, 1.33 gm, 0.055 moles) and benzyl thiol (6.57 gm, 0.053 moles) were added. The reaction mixture was stirred at 20-25° C. under nitrogen for 2 hour. Progress of the reaction was monitored by TLC. The reaction mixture was cooled to 0-10° C., added slowly DI water (200 ml). The solid product was filtered off, washed with water (50 ml). Poured hexane (100 ml) to the above crude product and stirred the suspension for 1 hour at 20-25° C. The solid product obtained was filtered off, washed with hexane (50 ml) and dried to yield the product, 5-(5-(benzylsulfanyl)-1-methyl-1H-pyrazol-4-yl)-2-methyl-2H-tetrazole at 40-45° C.

| Dry wt | 13 gm |
|---|---|
| Yield | 1.3 w/w (65%) |
| HPLC purity | 99.26% |

### Step 7: 1-methyl-4-(2-methyl-2H-tetrazol-5-yl)-1H-pyrazole-5-sulfonyl chloride

To a mixture of 5-(5-(benzylsulfanyl)-1-methyl-1H-pyrazol-4-yl)-2-methyl-2H-tetrazole obtained in step 6 (9 gm, 0.0314 moles), and aqueous acetic acid (10%, 50 ml) was slowly purged chlorine gas at 0-10° C. The progress of the reaction was monitored by TLC and HPLC. To the reaction mixture DI water (100 ml) was slowly, the solid obtained was filtered off, washed with water (50 ml) and dried at 60-65° C. to obtain 1-methyl-4-(2-methyl-2H-tetrazol-5-yl)-1H-pyrazole-5-sulfonyl chloride.

| | |
|---|---|
| Dry wt | 7.5 gm |
| Yield | 0.83 w/w (91%) |
| HPLC purity | 97.65% |
| MP | 97-100° C. |

### Step 8: 1-methyl-4-(2-methyl-2H-tetrazol-5-yl)-1H-pyrazole-5-sulfonamide

1-methyl-4-(2-methyl-2H-tetrazol-5-yl)-1H-pyrazole-5-sulfonyl chloride (6.8 gm, 0.026 moles) obtained in step 7, ethylene dichloride (30 ml) were taken. 25% of aqueous ammonia (7 ml, 0.133 moles) was slowly added to the reaction mixture and stirred at 20-30° C. for 1-2 hours. Progress of the reaction was monitored by HPLC. The excess solvent was recovered at reduced pressure. The mixture was cooled to −5 to 0° C. Poured DI water (20 ml) to the above residue and stirred the mixture for 30 min to 1 hour at −5 to 0° C. The solid product obtained was filtered, washed with cold DI water (70 ml) and dried at 60-65° C. to obtain 1-methyl-4-(2-methyl-2H-tetrazol-5-yl)-1H-pyrazole-5-sulfonamide.

| | |
|---|---|
| Dry wt | 6 gm |
| Yield | 0.85 w/w (96%) |
| HPLC purity | 99.89% |
| MP | 137-139° C. |

### Step 9: Azimsulfuron

A mixture of 1-methyl-4-(2-methyl-2H-tetrazol-5-yl)-1H-pyrazole-5-sulfonamide (5.8 gm, 0.0238 moles) obtained in step 8, phenyl (4,6-dimethoxypyrimidin-2-yl)carbamate (7.2 gm, 0.026 moles), and triethyl amine (3.58 gm, 0.035 moles) were taken in acetonitrile (50 ml) and stirred at 20-30° C. for 1 hour. Progress of the reaction mixture was monitored by HPLC. To the reaction mixture DI water (60 ml) was added, cooled the mixture to 0-10° C. and slowly added conc. HCl (34-37%, 6 ml) to adjust acidic pH. Stirred the reaction mixture at 20-30° C. for 1-2 hour, solid product was filtered off and washed with DI water (60 ml) and with IPA (20 ml) and dried at 60-65° C. to get Azimsulfuron.

| | |
|---|---|
| Dry wt | 9.5 gm |
| Yield | 1.64 w/w (94%) |
| HPLC purity | 99.45 |
| MP | 173-176° C. |

### Example 2

### Preparation of Azimsulfuron

### Step 1: 1-methyl-1H-pyrazole-4-carbaldehyde

1-methyl-1H-pyrazole-4-carbaldehyde was synthesized according to the method described in

### Step 1 of Example 1

### Step 2: 1-methyl-1H-pyrazole-4-carbaldehyde oxime

1-methyl-1H-pyrazole-4-carbaldehyde oxime was synthesized according to the method described in Step 2 of Example 1

### Step 3: 5-(1-methyl-1H-pyrazol-4-yl)-2H-tetrazole

5-(1-methyl-1H-pyrazol-4-yl)-2H-tetrazole was synthesized according to the method described in Step 3 of Example 1

### Step 4: 2-methyl-5-(1-methyl-1H-pyrazol-4-yl)-2H-tetrazole

2-methyl-5-(1-methyl-1H-pyrazol-4-yl)-2H-tetrazole was synthesized according to the method described in Step 4 of Example 1

### Step 5: 5-(5-chloro-1-methyl-1H-pyrazol-4-yl)-2-methyl-2H-tetrazole

5-(5-chloro-1-methyl-1H-pyrazol-4-yl)-2-methyl-2H-tetrazole was synthesized according to the method described in Step 5 of Example 1

### Step 6: Synthesis of (1-methyl-4-(2-methyl-2H-tetrazol-5-yl)-1H-pyrazole-5-thiol) or (1,2-bis(1-methyl-4-(2-methyl-2H-tetrazol-5-yl)-1H-pyrazol-5-yl)disulfane) or mixture thereof

To the above 5-(5-chloro-1-methyl-1H-pyrazole-4-yl)-2-methyl-2H-tetrazole), obtained from Step 5 (6 gm), added DMF (30 ml), sodium hydrosulfide (8 gm). The reaction mixture was heated at 100-120° C. under nitrogen for 10-14 hour. Progress of the reaction was monitored by HPLC. The reaction mixture was cooled to 0-10° C. and slowly added dil. HCl (1N) to make acidic pH. Ethyl acetate layer was partitioned and washed with brine (10%, 30 ml). Thereafter, ethyl acetate was distilled off at 50° C. at reduced pressure to obtain (1-methyl-4-(2-methyl-2H-tetrazol-5-yl)-1H-pyrazole-5-thiol) or (1,2-bis(1-methyl-4-(2-methyl-2H-tetrazol-5-yl)-1H-pyrazol-5-yl)disulfane) or mixture thereof.

| Dry weight | 5.5 gm |
|---|---|
| Yield | 0.96 w/w (98%) |
| Purity | 95% |

### Step 7: 1-methyl-4-(2-methyl-2H-tetrazol-5-yl)-1H-pyrazole-5-sulfonyl chloride

1-methyl-4-(2-methyl-2H-tetrazol-5-yl)-1H-pyrazole-5-sulfonyl chloride was synthesized according to the method described in Step 7 of Example 1

### Step 8: 1-methyl-4-(2-methyl-2H-tetrazol-5-yl)-1H-pyrazole-5-sulfonamide

1-methyl-4-(2-methyl-2H-tetrazol-5-yl)-1H-pyrazole-5-sulfonamide was synthesized according to the method described in Step 8 of Example 1

### Step 9: Azimsulfuron

Azimsulfuron was synthesized according to the method described in Step 9 of Example 1.

### Example 3

### Preparation of Azimsulfuron

### Step 1: 2-(ethoxymethylene)malononitrile

A mixture of triethyl orthoformate (67.3 gm, 0.454 moles) and malononitrile (20.0 gm, 0.302 moles) in acetic anhydride (77.2 gm, 0.75 moles) was refluxed for 4-5 hours at 110-140° C. The reaction was monitored by GC. Reaction mixture was cooled to room temperature. Concentrated the reaction mixture at 70° C. at reduced pressure to yield crude solid product, 2-(ethoxymethylene)malononitrile. The crude product was further purified either by vacuum distillation to yield pure product, 2-(ethoxymethylene)malononitrile.

| | |
|---|---|
| Dry wt | 36.0 gm |
| Yield | 1.80 w/w (98%) |
| HPLC purity | 99% |
| MP | 65-67° C. |

### Step 2: 5-amino-1-methyl-1H-pyrazole-4-carbonitrile

A solution of 2-(ethoxymethylene)malononitrile (20.0 gm, 0.163 moles) in methanol (80.0 ml) was slowly added to a mixture of N-methyl hydrazine sulfate (23.5 gm, 0.163 moles), triethyl amine (33.0 gm, 0.326 moles) and methanol (30.0 gm). The reaction mixture was refluxed for 1-2 hour. The progress of the reaction was monitored by HPLC. Excess methanol was distilled out at reduced pressure. To the above residue distilled water (40.0 ml) was added and the reaction mass was cooled to 0-15° C. 15% (w/v) aqueous sodium hydroxide solution (18.0 ml) was added to the above reaction mixture and the reaction mixture thereto was stirred for 30-45 min at 0-5° C., filtered, washed with cold water (400 ml) and air dried to yield 5-amino-1-methyl-1H-pyrazole-4-carbonitrile at 50-75° C.

| | |
|---|---|
| Dry wt | 15.4 gm |
| Yield | 0.77 w/w (77%) |
| HPLC purity | 98.67% |
| MP | 216-221° C. |

### Step 3: 5-chloro-1-methyl-1H-pyrazole-4-carbonitrile

A mixture of 5-amino-1-methyl-1H-pyrazole-4-carbonitrile (12.1 gm, 0.099 moles), hydrochloric acid (72.6 ml, strength: 35-37%) in DM water (60.0 ml) was stirred at room temperature. Reaction mixture was cooled to 0 to 5° C. and sodium nitrite solution (by dissolving sodium nitrite (8.2 gm, 0.119 moles) in DI water (240 ml)) was slowly added. The reaction mixture was stirred for 1-1.5 hours at 0 to 5° C. Urea solution [prepared by dissolving urea (1.2 gm) in DI water (6.0 ml)] is added slowly to the reaction mixture. Solid cuprous chloride (12.8 gm, 0.129 moles) was added to the above reaction mixture, stirred the reaction mixture for 3-4 hours at 0 to 5° C. Then the reaction temperature was raised to room temperature and stirred for 12-18 hours. Progress of the reaction was monitored by HPLC. To the above reaction mixture was added DI water (120 ml) and 25% (w/v) aqueous sodium hydroxide solution (80.0 ml) to adjust the pH to 3-3.5. 1,2-dichloroethane was poured to the reaction mixture and stirred for 10-15 min at 45-50° C. The inorganic salt was filtered off through hyflo bed. The organic layer was separated and aqueous layer was extracted with 1,2-dichloroethane. The entire organic layer was transferred to the reaction vessel and washed with 10% brine solution (50.0 ml). The organic layer was evaporated to obtain the 5-chloro-1-methyl-1H-pyrazole-4-carbonitrile.

| | |
|---|---|
| Dry wt | 11.8 gm |
| Yield | 0.97 w/w (84%) |
| HPLC purity | 93.34% |
| MP | 55-60° C. |

### Step 4: 5-(5-chloro-1-methyl-1H-pyrazole-4-yl)-2H-tetrazole

The above product, 5-chloro-1-methyl-1H-pyrazole-4-carbonitrile (13.0 gm, 0.092 moles), was dissolved in DMF (40 ml), followed by addition of ammonium chloride (9.9 gm, 0.184 moles) and sodium azide (12.0 gm, 0.184 moles) and stirred at 100-140° C. for 10-16 hours. The progress of the reaction was monitored by HPLC. The reaction mixture was cooled at 0-5° C. and quenched with aqueous HCl (78.0 ml) and stirred for 30-60 min at 0-5° C. The solid material obtained was filtered, washed with cold DM water (40 ml) and dried at 60-65° C. to yield the product, 5-(5-chloro-1-methyl-1H-pyrazole-4-yl)-2H-tetrazole

| | |
|---|---|
| Dry wt | 16.2 gm |
| Yield | 1.25 w/w (96%) |
| HPLC purity | 99.26% |
| MP | 215-219° C. |

### Step 5: 5-(5-chloro-1-methyl-1H-pyrazole-4-yl)-2-methyl-2H-tetrazole), 5-(5-chloro-1-methyl-1H-pyrazole-4-yl)-1-methyl-2H-tetrazole), or mixtures thereof

The above product of step-4, 5-(5-chloro-1-methyl-1H-pyrazole-4-yl)-2H-tetrazole (10.0 gm, 0.0541 moles), was dissolved in acetone (50.0 ml). Potassium carbonate (6.0 gm, 0.0433 moles) was added and stirred for about 1 hour at 20-30° C. To this mixture, slowly added a solution of DMS (9.5 gm, 0.0757 moles) in acetone (10.0 ml) maintaining the temperature below 30° C. The reaction mixture was stirred at room temperature for about 1 hour and monitored the progress of the reaction by HPLC. The inorganic solids were filtered off Acetone was distilled off from the filtrate to obtain the crude product containing 5-(5-chloro-1-methyl-1H-pyrazole-4-yl)-2-methyl-2H-tetrazole), its regio-isomer, 5-(5-chloro-1-methyl-1H-pyrazole-4-yl)-1-methyl-2H-tetrazole), or mixture thereof. The desired regio-isomer, 5-(5-chloro-1-methyl-1H-pyrazol-4-yl)-2-methyl-2H-tetrazole), was isolated by column chromatography.

| | |
|---|---|
| Dry wt | 7.81 gm |
| Yield | 0.78 w/w (73%) |
| HPLC purity | 99.12% |
| MP | 125-127° C. |

### Step 6: 5-(5-(benzylsulfanyl)-1-methyl-1H-pyrazol-4-yl)-2-methyl-2H-tetrazole

Product of Step 6, 5-(5-(benzylsulfanyl)-1-methyl-1H-pyrazol-4-yl)-2-methyl-2H-tetrazole was synthesized according to method described in Step-6 of Example 1.

### Step 7: 1-methyl-4-(2-methyl-2H-tetrazol-5-yl)-1H-pyrazole-5-sulfonyl chloride

1-methyl-4-(2-methyl-2H-tetrazol-5-yl)-1H-pyrazole-5-sulfonyl chloride was synthesized according to the method described in Step 7 of Example 1

### Step 8: 1-methyl-4-(2-methyl-2H-tetrazol-5-yl)-1H-pyrazole-5-sulfonamide

1-methyl-4-(2-methyl-2H-tetrazol-5-yl)-1H-pyrazole-5-sulfonamide was prepared according to the method described in Step 8 of Example 1.

### Step 9: Azimsulfuron

Azimsulfuron was prepared according to the method described in Step 9 of Example 1.

### Example 4

### Preparation of azimsulfuron

### Step 1: 2-(ethoxymethylene)malononitrile

The preparation of 2-(ethoxymethylene)malononitrile was carried out in a manner similar to that described in Step 1 of Example 3.

### Step 2: 5-amino-1-methyl-1H-pyrazole-4-carbonitrile

The 5-amino-1-methyl-1H-pyrazole-4-carbonitrile was synthesized according to the method described in Step 2 of Example 3.

### Step 3: 5-chloro-1-methyl-1H-pyrazole-4-carbonitrile

The preparation of 5-chloro-1-methyl-1H-pyrazole-4-carbonitrile was carried out in a manner similar to that described in Step 3 of Example 3.

### Step 4: 5-(5-chloro-1-methyl-1H-pyrazole-4-yl)-2H-tetrazole

The preparation of 5-(5-chloro-1-methyl-1H-pyrazole-4-yl)-2H-tetrazole was carried out in a manner similar to that described in Step 4 of Example 3.

### Step 5: 5-(5-chloro-1-methyl-1H-pyrazole-4-yl)-2-methyl-2H-tetrazole), 5-(5-chloro-1-methyl-1H-pyrazole-4-yl)-1-methyl-2H-tetrazole), or mixtures thereof

The 5-(5-chloro-1-methyl-1H-pyrazole-4-yl)-2-methyl-2H-tetrazole) or its regio-isomer 5-(5-chloro-1-methyl-1H-pyrazole-4-yl)-1-methyl-2H-tetrazole), or mixtures thereof were synthesized according to the method described in Step 5 of Example 3.

### Step 6: Synthesis of (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof

Synthesis of (1-methyl-4-(2-methyl-2H-tetrazol-5-yl)-1H-pyrazole-5-thiol) or (1,2-bis(1-methyl-4-(2-methyl-2H-tetrazol-5-yl)-1H-pyrazol-5-yl)disulfane) or mixture thereof was synthesized according to the method described in Step 6 of Example 2.

### Step 7: 1-methyl-4-(2-methyl-2H-tetrazol-5-yl)-1H-pyrazole-5-sulfonyl chloride

1-methyl-4-(2-methyl-2H-tetrazol-5-yl)-1H-pyrazole-5-sulfonyl chloride was synthesized according to the method described in Step 7 of Example 1.

### Step 8: 1-methyl-4-(2-methyl-2H-tetrazol-5-yl)-1H-pyrazole-5-sulfonamide

The 1-methyl-4-(2-methyl-2H-tetrazol-5-yl)-1H-pyrazole-5-sulfonamide was synthesized according to the method described in Step 8 of Example 1.

### Step 9: Azimsulfuron

The preparation of azimsulfuron was carried out in a similar manner to that described in Step 9 of Example 1.

### Example 5

### Preparation of Azimsulfuron

### Step 1: 2-(ethoxymethylene)malononitrile

The preparation 2-(ethoxymethylene)malononitrile was carried out in a manner similar to that described in Step 1 of Example 3.

### Step 2: 5-amino-1-methyl-1H-pyrazole-4-carbonitrile

The 5-amino-1-methyl-1H-pyrazole-4-carbonitrile was synthesized according to the above method described in Step 2 of Example 3.

### Step 3: 5-bromo-1-methyl-1H-pyrazole-4-carbonitrile

A mixture of 5-amino-1-methyl-1H-pyrazole-4-carbonitrile (20 gm, 1.63 moles) obtained from Step 2, hydrobromic acid (280 ml, strength: 48%) in DM water (605 ml) was stirred at 0 to 5° C. and sodium nitrite solution (by dissolving sodium nitrite (16.94 gm, 0.245 moles) in DI water (242 ml)) was slowly added. Solid cuprous bromide (37.22 gm, 0.260 moles) was added to the above reaction mixture, stirred the reaction mixture for 3-4 hours at 0 to 5° C. Then the reaction temperature was raised to room temperature and stirred for 15-18 hours. To the above reaction mixture cold DI water (1000 ml) was added at 0-5° C. and stirred the resulting suspension for 1-2 hours. Progress of the reaction was monitored by HPLC. The solid product was filtered, washed with cold DI water (200 ml) and dried at 60-65° C. to yield the product, 5-bromo-1-methyl-1H-pyrazole-4-carbonitrile.

| | |
|---|---|
| Dry wt | 22 gm |
| Yield | 1.1 w/w (72%) |
| HPLC purity | 99.58% |
| MP | 90-93° C. |

### Step 4: 5-(5-bromo-1-methyl-1H-pyrazole-4-yl)-2H-tetrazole

The 5-bromo-1-methyl-1H-pyrazole-4-carbonitrile (14.85 gm, 0.0798 moles), was dissolved in toluene (148 ml), and tri-n-butyl tin chloride (64.83 gm, 0.199 moles) and sodium azide (12.93 gm, 0.199 moles) was added to it. The reaction mixture was stirred at 100-145° C. for 7-8 hours. The progress of the reaction was monitored by HPLC. The reaction mixture was cooled at 10-15° C. and 10% ethanolic HCl solution (100 ml) was added and stirred for 1-1.5 hours at 10-15° C. The inorganic salt was filtered off, washed with ethanol (30 ml). Mother liquor and washings were collected and excess of toluene and ethanol was recovered at 50-55° C. at reduced pressure. Poured di-isopropyl ether (100 ml) to the above residue and stirred the resulting suspension for 1-2 hours at 20-25° C. The solid product was filtered, washed with di-isopropyl ether (30 ml) and dried at 60-65° C. to yield the product, 5-(5-bromo-1-methyl-1H-pyrazol-4-yl)-2H-tetrazole.

| | |
|---|---|
| Dry wt | 17 gm |
| Yield | 1.14 w/w (93%) |
| HPLC purity | 99.72% |
| MP | 202-207° C. |

### Step 5: 5-(5-bromo-1-methyl-1H-pyrazole-4-yl)-2-methyl-2H-tetrazole

Dissolve 5-(5-bromo-1-methyl-1H-pyrazol-4-yl)-2H-tetrazole (Ic) (9.5 gm, 0.0414 moles) in acetonitrile (100 ml). To this mixture, potassium hydroxide (5.71 gm, 0.10 moles) and methyl iodide (24.5 gm, 0.171 moles) was added and stirred at 20-30° C. for 20-25 hours. The progress of the reaction was monitored by HPLC. The acetonitrile was recovered at 55-60° C. at reduced pressure. To the above residue dichloromethane (100 ml) and DI water (100 ml) was added and stirred the above reaction mixture for 10-15 min at 20-25° C. The reaction mixture was allowed to settle. The organic layer was separated and aqueous layer was extracted with DCM. The entire organic layer was transferred to the reaction vessel and washed with 10% brine solution (50 ml). Stirred for 10-15 min and separated the final aqueous layer and organic layer. The organic layer was evaporated to obtain the crude product. 5-(5-bromo-1-methyl-1H-pyrazole-4-yl)-2-methyl-2H-tetrazole) was obtained by purifying crude product by column chromatography by eluting ethyl acetate/hexane (50:50)

| Dry wt | 5 gm |
|---|---|
| Yield | 0.52 w/w (50%) |
| HPLC purity | 99.95% |

### Step 6: 5-(5-(benzylsulfanyl)-1-methyl-1H-pyrazol-4-yl)-2-methyl-2H-tetrazole

A mixture of 5-(5-bromo-1-methyl-1H-pyrazol-4-yl)-2-methyl-2H-tetrazole (17.7 gm, 0.072 moles), sodium hydride (2.59 gm, 0.108 moles), benzyl thiol (9.89 gm, 0.079 moles) and DMF (80 ml) was stirred at 60-80° C. for 1 hour. The progress of the reaction was monitored by HPLC and TLC. The reaction mixture was cooled at 0-10° C. and slowly added DI water (200 ml). The solid product was filtered off, washed with water (50 ml and hexane (100 ml) to afforded the product, 5-(5-(benzylsulfanyl)-1-methyl-1H-pyrazol-4-yl)-2-methyl-2H-tetrazole.

| Dry wt | 16 gm |
|---|---|
| Yield | 0.91 w/w (78%) |
| HPLC purity | 99.43% |

### Step 7: 1-methyl-4-(2-methyl-2H-tetrazol-5-yl)-1H-pyrazole-5-sulfonyl chloride

The preparation 1-methyl-4-(2-methyl-2H-tetrazol-5-yl)-1H-pyrazole-5-sulfonyl chloride was carried out in a similar manner to that described in Step 7 of Example 1.

### Step 8: 1-methyl-4-(2-methyl-2H-tetrazol-5-yl)-1H-pyrazole-5-sulfonamide

The 1-methyl-4-(2-methyl-2H-tetrazol-5-yl)-1H-pyrazole-5-sulfonamide was synthesized according to the method described in Step 8 of Example 1.

### Step 9: Azimsulfuron

The preparation of azimsulfuron was carried out in a similar manner to that described in Step 9 of Example 1.

### Example 6

### Preparation of Azimsulfuron

### Step 1: 4-iodo-1-methyl-1H-pyrazole

A mixture of 1-methyl-1H-pyrazole (7) (37 gm, 0.6090 moles), iodine (57.1 gm, 0.225 moles), hydrogen peroxide (9.2 gm, 0.27 moles) and DI water (110 ml) were stirred for 24 hour at 20-30° C. Progress of the reaction was monitored by HPLC and TLC. The reaction mixture was cooled to 5-10° C. The reaction mixture was quenched with 20% aqueous sodium bisulfite (100 ml) and stirred for 1 hour at 5-10° C. The solid product obtained was filtered off, washed with cold DI water (100 ml) and dried at 40-45° C. to yield the product, 4-iodo-1-methyl-1H-pyrazole.

| | |
|---|---|
| Dry wt | 56 gm |
| Yield | 1.51 w/w (60%) |

### Step 2: 1-methyl-1H-pyrazole-4-carbonitrile

To a solution of 4-iodo-1-methyl-1H-pyrazole (55 gm, 0.264 moles) in N,N-dimethylacetamide (100 ml) was added potassium ferrocyanide (24.5 gm, 0.058 moles), palladium (II) acetate (0.592 gm, 0.0026 moles) and sodium carbonate (27.98 gm, 0.264 moles). The reaction mixture was evacuated and backfilled with nitrogen (3 times). The mixture was stirred for 12 hour at 90-110° C. Progress of the reaction was monitored by HPLC. The reaction mixture was cooled to 20-30° C., to this added DI water (500 ml), ethyl acetate (500 ml) and stirred for 1 hour at 20-30° C. The reaction mixture was filtered through pad of celite. The organic layer was separated and aqueous layer was extracted with ethyl acetate (200 ml), stirred for 15 min and separated the final aqueous layer and organic layer. The organic layer was washed with brine solution (200 ml). Ethyl acetate was recovered at reduced pressure at 60-70° C. The mixture was degassed for 2 hour at reduced pressure at 60-70° C., cooled the mixture to 20-30° C. Hexane (400 ml) was added to the mixture and stirred for 1 hour at 20-30° C. The solid product obtained was filtered off, washed with cold DI water (100 ml) and dried at 40-50° C. to yield the product, 1-methyl-1H-pyrazole-4-carbonitrile.

| | |
|---|---|
| Dry wt | 17.94 gm |
| Yield | 0.32 w/w (63%); |

### Step 3: 5-(1-methyl-1H-pyrazol-4-yl)-2H-tetrazole

1-methyl-1H-pyrazole-4-carbonitrile (18 gm, 0.168 moles) obtained in step 2, tri-n-butyl tin chloride (65 gm, 0.218 moles), sodium azide (14 gm, 0.201 moles) were taken in toluene (180 ml). The reaction mixture was stirred for 24 hours at 110-120° C. The progress of the reaction was monitored by HPLC. The above reaction mixture was cooled to 10-15° C., to this added 10% ethanolic HCl solution (100 ml, pH=1-2). The reaction mixture was stirred for 1-1.5 hours at 10-15° C., filtered off the inorganic salts, washed with ethanol (30 ml). The mother liquor and washings were collected and toluene and ethanol was recovered at 50-55° C. at reduced pressure. To the above residue at 20-25° C., added di-isopropyl ether (100 ml), stirred for 1-2 hours at 20-25° C., filtered the solid, washed with di-isopropyl ether (30 ml) and dried at 60-65° C. to yield the product, 5-(1-methyl-1H-pyrazol-4-yl)-2H-tetrazole.

| | |
|---|---|
| Dry wt | 25 gm |
| Yield | 1.4 w/w (99%) |

### Step 4: 2-methyl-5-(1-methyl-1H-pyrazol-4-yl)-2H-tetrazole

The preparation of 2-methyl-5-(1-methyl-1H-pyrazol-4-yl)-2H-tetrazole was carried out in a manner similar to that described in Step 4 of Example 1.

### Step 5: 5-(5-chloro-1-methyl-1H-pyrazol-4-yl)-2-methyl-2H-tetrazole

The 5-(5-chloro-1-methyl-1H-pyrazol-4-yl)-2-methyl-2H-tetrazole was synthesized according to the method described in Step 5 of Example 1.

### Step 6: 5-(5-(benzylsulfanyl)-1-methyl-1H-pyrazol-4-yl)-2-methyl-2H-tetrazole

5-(5-(Benzylsulfanyl)-1-methyl-1H-pyrazol-4-yl)-2-methyl-2H-tetrazole was synthesized according to the method described in Step 5 of Example 1.

### Step 7: 1-methyl-4-(2-methyl-2H-tetrazol-5-yl)-1H-pyrazole-5-sulfonyl chloride

The preparation 1-methyl-4-(2-methyl-2H-tetrazol-5-yl)-1H-pyrazole-5-sulfonyl chloride was carried out in a similar manner to that described in Step 7 of Example 1.

### Step 8: 1-methyl-4-(2-methyl-2H-tetrazol-5-yl)-1H-pyrazole-5-sulfonamide

The 1-methyl-4-(2-methyl-2H-tetrazol-5-yl)-1H-pyrazole-5-sulfonamide was synthesized according to the method described in Step 8 of Example 1.

### Step 9: Azimsulfuron

The preparation of azimsulfuron was carried out in a similar manner to that described in Step 9 of Example 1.

### Example 7

### Preparation of Azimsulfuron

### Step 1: 4-iodo-1-methyl-1H-pyrazole

The preparation of 4-iodo-1-methyl-1H-pyrazole was carried out in a similar manner to that described in Step 1 of Example 6.

### Step 2: 1-Methyl-1H-pyrazole-4-carbonitrile

The preparation of 1-Methyl-1H-pyrazole-4-carbonitrile was carried out in a similar manner to that described in Step 2 of Example 6.

### Step 3: 5-(1-methyl-1H-pyrazol-4-yl)-2H-tetrazole

The preparation of 5-(1-methyl-1H-pyrazol-4-yl)-2H-tetrazole was carried out in a similar manner to that described in Step 3 of Example 6.

### Step 4: 2-methyl-5-(1-methyl-1H-pyrazol-4-yl)-2H-tetrazole

The preparation of 2-methyl-5-(1-methyl-1H-pyrazol-4-yl)-2H-tetrazole was carried out in a manner similar to that described in Step 4 of Example 1.

### Step 5: 5-(5-chloro-1-methyl-1H-pyrazol-4-yl)-2-methyl-2H-tetrazole

The 5-(5-chloro-1-methyl-1H-pyrazol-4-yl)-2-methyl-2H-tetrazole was synthesized according to the method described in Step 5 of Example 1.

### Step 6: Synthesis of (1-methyl-4-(2-methyl-2H-tetrazol-5-yl)-1H-pyrazole-5-thiol) or its disulfide (1,2-bis-(1-methyl-4-(2-methyl-2H-tetrazol-5-yl)-1H-pyrazol-5-yl)disulfane), or mixture thereof

Synthesis of (1-methyl-4-(2-methyl-2H-tetrazol-5-yl)-1H-pyrazole-5-thiol) or (1,2-bis(1-methyl-4-(2-methyl-2H-tetrazol-5-yl)-1H-pyrazol-5-yl)disulfane) or mixture thereof was synthesized according to the method described in Step 6 of Example 2.

### Step 7: 1-methyl-4-(2-methyl-2H-tetrazol-5-yl)-1H-pyrazole-5-sulfonyl chloride

The preparation 1-methyl-4-(2-methyl-2H-tetrazol-5-yl)-1H-pyrazole-5-sulfonyl chloride was carried out in a similar manner to that described in Step 7 of Example 1.

### Step 8: 1-methyl-4-(2-methyl-2H-tetrazol-5-yl)-1H-pyrazole-5-sulfonamide

The 1-methyl-4-(2-methyl-2H-tetrazol-5-yl)-1H-pyrazole-5-sulfonamide was synthesized according to the method described in Step 8 of Example 1.

Azimsulfuron prepared by the process of the present disclosure may be in the form of a white crystalline solid free from visible extraneous matter, particulate solids, and granules of varying particle size, wettable powders or dusts.

The compound can also be formulated as water soluble wettable powders (WP), water dispersible granules (WDG), water emulsifiable granules (WEG), suspeoemulsion (SE) or oil suspension concentrate (SC). It may be applied to a field in the form of a liquid spray, or as solid powder or granules.

Although the subject matter has been described in considerable detail with reference to certain preferred embodiments thereof, other embodiments are possible. As such, the spirit and scope of the present disclosure should not be limited to the description of the preferred embodiment contained therein.

1. A process for preparation of azimsulfuron or its salts, isomers, and other derivatives thereof, comprising:
treating a compound of formula I, or mixtures thereof, 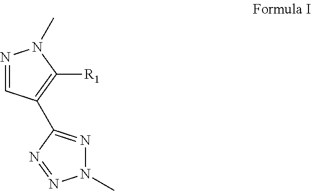
wherein,
R₁ is selected from —SH, —SCH₂Ph, —SR or —S—SR₁′; where R is a straight or a branched chain C₁₋₄ alkyl and R₁′ is a group of formula II where * denotes the point of attachment 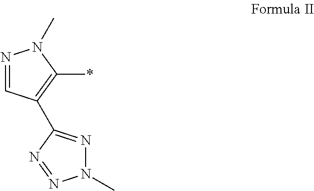 [SMILES: *c1c(-c2nnn(C)n2)cnn1C]
with aqueous acetic acid or formic acid, chlorine gas or sodium hypochlorite in presence of hydrochloric acid and chlorinated solvents such as dichloromethane, 1,2-dichloroethane or N-chlorosuccinimide in presence of aq. acetic acid or hydrogen peroxide and hydrochloric acid in aqueous cyclic ether such as tetrahydrofuran or 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and a solvent selected from a chlorinated hydrocarbon, such as dichloromethane, 1,2-dichloroethane or chloroform; a cyclic ether such as tetrahydrofuran, or 1,4-dioxane; or a ketone such as acetone, ethyl methyl ketone or methyl isobutyl ketone; and
treating 1-methyl-4-(2-methyl-2H-tetrazol-5-yl)-1H-pyrazole-5-sulfonamide with phenyl (4,6-dimethoxypyrimidin-2-yl)carbamate in the presence of an organic base and a polar solvent to obtain azimsulfuron or its salts, isomers, and other derivatives thereof.

2. The process as claimed in claim 1, wherein the oxidizing agent is selected from hydrogen peroxide, sodium hypochlorite, N-chlorosuccinimide or chlorine gas.

3. The process as claimed in claim 1, wherein the polar solvent is selected from N,N-dimethylformamide, N,N-dimethylacetamide, N-methyl-2-pyrrolidone, acetonitrile, acetone, dichloromethane, 1,2-dichloroethane, methanol, tetrahydrofuran, or water; and the organic base is selected from di-isopropyl ethyl amine, di-isopropyl amine, tri-n-butyl amine, triethyl amine, trimethyl amine or pyridine.

4. The process as claimed in claim 1, comprising:
treating a compound of formula I, or mixture thereof 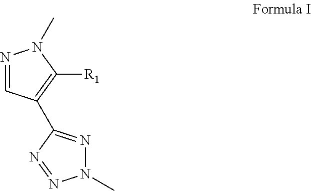
wherein, R₁ is selected from —SH, —SCH₂Ph, —SR or —S—SR₁′; where R is a straight or a branched chain C₁₋₄ alkyl and R₁′ is a group of formula II where * denotes the point of attachment 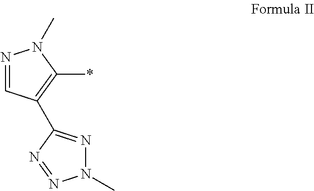 [SMILES: *c1c(-c2nnn(C)n2)cnn1C]
with aqueous acetic acid or formic acid, chlorine gas and sodium hypochlorite in presence of hydrochloric acid and chlorinated solvents such as dichloromethane, 1,2-dichloroethane or hydrogen peroxide and hydrochloric acid in aqueous cyclic ether such as tetrahydrofuran or 1,4-dioxane, to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

5. The process as claimed in claim 1, wherein the compound of formula I is obtained by treating a 5-(5-halo-1-methyl-1H-pyrazole-4-yl)-2-methyl-2H-tetrazole or 5-(5-halo-1-methyl-1H-pyrazole-4-yl)-1-methyl-2H-tetrazole or mixture thereof with a thiol or an alkali metal hydrosulfide in presence of a solvent selected from N,N-dimethylformamide, N,N-dimethylacetamide, N-methyl-2-pyrrolidone, acetonitrile, acetone, tetrahydrofuran or water.

6. The process as claimed in claim 1, wherein the compound of formula I
wherein R₁ is selected from —SH, or —S—SR₁′; where R₁′ is a group of formula II where * denotes the point of attachment 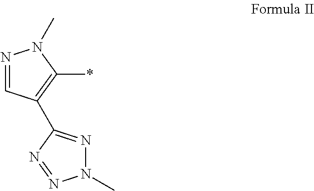 [SMILES: *c1c(-c2nnn(C)n2)cnn1C]
is obtained by treating a 5-(5-halo-1-methyl-1H-pyrazole-4-yl)-2-methyl-2H-tetrazole with an alkali metal hydrosulfide in the presence of N,N-dimethylformamide

7. The process as claimed in claim 5, wherein the alkali metal hydrosulfide is selected from sodium hydrosulfide or potassium hydrosulfide.

8. The process as claimed in claim 1, wherein the compound of formula I wherein R₁ is selected from —SCH₂Ph or —SR where R is a straight or a branched chain C₁₋₄ alkyl; is obtained by treating 5-(5-halo-1-methyl-1H-pyrazole-4-yl)-2-methyl-2H-tetrazole with a thiol in the presence of sodium hydride and N,N-dimethylformamide.

9. The process as claimed in claim 5, wherein the thiol is selected from C₁₋₄ alkyl thiol or benzyl thiol.

10. The process as claimed in claim 5, wherein the 5-(5-halo-1-methyl-1H-pyrazole-4-yl)-2-methyl-2H-tetrazole is obtained by halogenating 2-methyl-5-(1-methyl-1H-pyrazol-4-yl)-2H-tetrazole with a halogenating agent, selected from N-chlorosuccinimide, N-bromosuccinimide or N-iodosuccinimide in the presence of a solvent selected from N,N-dimethylformamide, N,N-dimethylacetamide, N-methyl-2-pyrrolidone, acetonitrile, acetone, dichloromethane, 1,2-dichloroethane, methanol, tetrahydrofuran, ethyl acetate, or butyl acetate.

11. The process as claimed in claim 5, wherein the 5-(5-halo-1-methyl-1H-pyrazole-4-yl)-2-methyl-2H-tetrazole, or 5-(5-halo-1-methyl-1H-pyrazole-4-yl)-1-methyl-1H-tetrazole, or mixtures thereof is obtained by methylating a 5-(5-halo-1-methyl-1H-pyrazole-4-yl)-2H-tetrazole with a methylating agent such as dimethyl sulfate, methyl iodide or methyl bromide, in presence of a base selected from an alkali metal hydroxide base such as sodium hydroxide, potassium hydroxide or lithium hydroxide; or an alkali metal carbonate base such as sodium carbonate, sodium bicarbonate, potassium carbonate or cesium carbonate; or an organic base such as triethyl amine, di-isopropyl amine, di-isopropyl ethyl amine, pyridine, pyrrolidine or N,N-dimethyl aniline, in the presence of a polar solvent such as N,N-dimethylformamide, N,N-dimethylacetamide, N-methyl-2-pyrrolidone, acetonitrile, acetone, dichloromethane, 1,2-dichloroethane, methanol, tetrahydrofuran or water.

12. The process as claimed in claim 10, wherein 2-methyl-5-(1-methyl-1H-pyrazol-4-yl)-2H-tetrazole is obtained by:
treating 1-methyl-1H-pyrazole with phosphoryl chloride in the presence of N,N-dimethylformamide, to obtain 1-methyl-1H-pyrazole-4-carbaldehyde;
treating 1-methyl-1H-pyrazole-4-carbaldehyde with hydroxylamine hydrochloride in the presence of ethanol, to obtain 1-methyl-1H-pyrazole-4-carbaldehyde oxime;
treating 1-methyl-1H-pyrazole-4-carbaldehyde oxime with sodium azide in the presence of ammonium chloride and N,N-dimethylformamide, to form 5-(1-methyl-1H-pyrazol-4-yl)-2H-tetrazole; and
reacting 5-(1-methyl-1H-pyrazole-4-yl)-2H-tetrazole with methyl iodide or dimethyl sulphate, in the presence of potassium hydroxide or potassium carbonate to obtain 2-methyl-5-(1-methyl-1H-pyrazol-4-yl)-2H-tetrazole.

13. The process as claimed in claim 10, wherein 2-methyl-5-(1-methyl-1H-pyrazol-4-yl)-2H-tetrazole (VII) is obtained by:
treating 1-methyl-1H-pyrazole with iodine and hydrogen peroxide in aqueous medium to obtain 4-iodo-1-methyl-1H-pyrazole;
treating 4-iodo-1-methyl-1H-pyrazole with potassium ferrocyanide and palladium (II) acetate in the presence of sodium carbonate, and N,N-dimethylacetamide, to obtain 1-methyl-1H-pyrazole-4-carbonitrile;
treating 1-methyl-1H-pyrazole-4-carbonitrile with sodium azide and either with tri-n-butyl tin chloride in the presence of toluene, or ammonium chloride in the presence of N,N-dimethylformamide to obtain 5-(1-methyl-1H-pyrazol-4-yl)-2H-tetrazole; and
reacting 5-(1-methyl-1H-pyrazole-4-yl)-2H-tetrazole with methyl iodide or dimethyl sulphate, in the presence of potassium hydroxide or potassium carbonate to obtain 2-methyl-5-(1-methyl-1H-pyrazol-4-yl)-2H-tetrazole.

14. The process as claimed in claim 5, wherein the 5-(5-halo-1-methyl-1H-pyrazole-4-yl)-2H-tetrazole is obtained by:
treating malononitrile with triethyl orthoformate in the presence of acetic anhydride to obtain 2-(ethoxymethylene)malononitrile;
treating 2-(ethoxymethylene)malononitrile with an N-methyl hydrazine sulfate or N-methyl hydrazine and a base selected from an alkali metal hydroxide base such as sodium hydroxide, potassium hydroxide or lithium hydroxide; or an alkali metal carbonate base such as sodium carbonate, sodium bicarbonate, potassium carbonate or cesium carbonate; or an organic base such as triethyl amine, di-isopropyl amine, di-isopropyl ethyl amine, pyridine, pyrrolidine or N,N-dimethyl aniline, in the presence of an alcohol such as methanol, ethanol or propanol to obtain 5-amino-1-methyl-1H-pyrazole-4-carbonitrile;
halogenating 5-amino-1-methyl-1H-pyrazole-4-carbonitrile with sodium nitrite and a cuprous halide such as cuprous chloride, cuprous bromide or cuprous iodide, with a mineral acid such as hydrochloric acid, hydrobromic acid or sulfuric acid, to obtain 5-halo-1-methyl-1H-pyrazole-4-carbonitrile; and
treating 5-halo-1-methyl-1H-pyrazole-4-carbonitrile with sodium azide in the presence of either an ammonium halide such as ammonium chloride, ammonium bromide or ammonium iodide, and a polar solvent such as N,N-dimethylformamide, N,N-dimethylacetamide, N-methyl-2-pyrrolidone, 1,2-dichloroethane, tetrahydrofuran or water, or tri-n-butyl tin halide such as tri-n-butyl tin chloride, tri-n-butyl tin bromide or tri-n-butyl tin iodide, and a non polar solvent such as toluene, xylene or benzene, to obtain 5-(5-halo-1-methyl-1H-pyrazole-4-yl)-2H-tetrazole).

15. The process as claimed in claim 1, comprising:
treating 5-(5-chloro-1-methyl-1H-pyrazole-4-yl)-2-methyl-2H-tetrazole with benzyl thiol in the presence of a N,N-dimethylformamide and sodium hydride to obtain compound 5-(5-(benzylthio)-1-methyl-1H-pyrazol-4-yl)-2-methyl-2H-tetrazole of formula I, wherein R₁ is —SCH₂Ph; 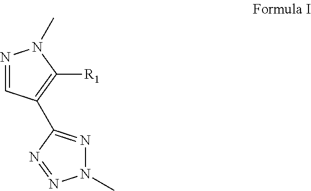
treating 5-(5-(benzylthio)-1-methyl-1H-pyrazol-4-yl)-2-methyl-2H-tetrazole of formula I with aqueous acetic acid or formic acid and chlorine gas or sodium hypochlorite in presence of
hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl) carbamate in the presence of triethylamine and acetonitrile to obtain azimsulfuron or its salts, isomers, and other derivatives thereof.

16. The process as claimed in claim 1, comprising:
treating 5-(5-chloro-1-methyl-1H-pyrazole-4-yl)-2-methyl-2H-tetrazole with sodium hydrosulfide in the presence of a N,N-dimethylformamide to obtain the compounds (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof, of the formula 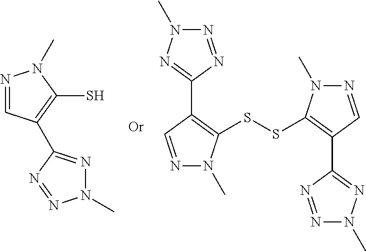
treating 1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof, with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

17. The process as claimed in claim 1, comprising:
chlorinating 2-methyl-5-(1-methyl-1H-pyrazole-4-yl)-2H-tetrazole with N-chlorosuccinimide, in the presence acetonitrile, to obtain 5-(5-chloro-1-methyl-1H-pyrazol-4-yl)-2-methyl-2H-tetrazole;
treating 5-(5-chloro-1-methyl-1H-pyrazole-4-yl)-2-methyl-2H-tetrazole with sodium hydrosulfide in the presence of a N,N-dimethylformamide to obtain the compounds (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof, of the formula 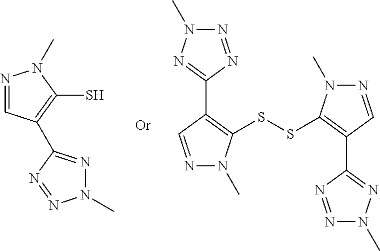
treating 1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof, with aqueous acetic acid or formic acid and chlorine gas or sodium hypochlorite in presence of
hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

18. The process as claimed in claim 1, comprising:
chlorinating 2-methyl-5-(1-methyl-1H-pyrazole-4-yl)-2H-tetrazole with N-chlorosuccinimide, in the presence acetonitrile, to obtain 5-(5-chloro-1-methyl-1H-pyrazol-4-yl)-2-methyl-2H-tetrazole;
treating 5-(5-chloro-1-methyl-1H-pyrazole-4-yl)-2-methyl-2H-tetrazole with benzyl thiol in the presence of a N,N-dimethylformamide and sodium hydride to obtain compound 545-(benzylthio)-1-methyl-1H-pyrazol-4-yl)-2-methyl-2H-tetrazole of formula I, wherein R₁ is —SCH₂Ph; 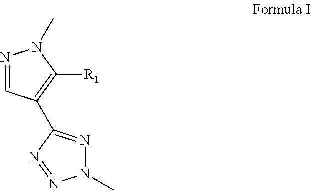
treating 5-(5-(benzylthio)-1-methyl-1H-pyrazol-4-yl)-2-methyl-2H-tetrazole of formula I with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

19. A process for preparation of azimsulfuron or its salts, isomers, and other derivatives thereof, comprising:
methylating 5-(5-chloro-1-methyl-1H-pyrazole-4-yl)-2H-tetrazole with a methylating agent such as dimethyl sulfate, methyl iodide or methyl bromide and potassium carbonate, in the presence of acetone to obtain 5-(5-chloro-1-methyl-1H-pyrazole-4-yl)-2-methyl-2H-tetrazole, or its regio-isomer 5-(5-chloro-1-methyl-1H-pyrazole-4-yl)-1-methyl-1H-tetrazole, or mixtures thereof;
treating 5-(5-chloro-1-methyl-1H-pyrazole-4-yl)-2-methyl-2H-tetrazole, or its regio-isomer 5-(5-chloro-1-methyl-1H-pyrazole-4-yl)-1-methyl-1H-tetrazole, or mixtures thereof with sodium hydrosulfide in the presence of N,N-dimethylformamide to obtain the compounds (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof, of the formula 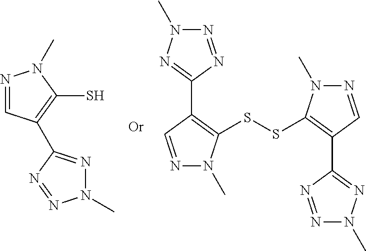
treating 1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof, with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

20. A process for preparation of azimsulfuron or its salts, isomers, and other derivatives thereof, comprising:
methylating 5-(5-chloro-1-methyl-1H-pyrazole-4-yl)-2H-tetrazole with a methylating agent such as dimethyl sulfate, methyl iodide or methyl bromide and potassium carbonate, in the presence of acetone to obtain 5-(5-chloro-1-methyl-1H-pyrazole-4-yl)-2-methyl-2H-tetrazole, or its regio-isomer 5-(5-chloro-1-methyl-1H-pyrazole-4-yl)-1-methyl-1H-tetrazole, or mixtures thereof;
treating 5-(5-chloro-1-methyl-1H-pyrazole-4-yl)-2-methyl-2H-tetrazole or its regio-isomer 5-(5-chloro-1-methyl-1H-pyrazole-4-yl)-1-methyl-2H-tetrazole or mixture thereof with benzyl thiol in the presence of a N,N-dimethylformamide and sodium hydride to obtain compound 5-(5-(benzylthio)-1-methyl-1H-pyrazol-4-yl)-2-methyl-2H-tetrazole of formula I, wherein R₁ is —SCH₂Ph; 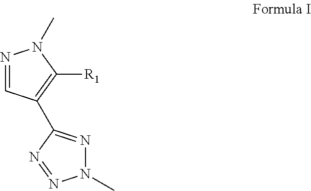
treating 5-(5-(benzylthio)-1-methyl-1H-pyrazol-4-yl)-2-methyl-2H-tetrazole of formula I with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

21. The process as claimed in claim 1, comprising:
treating 1-methyl-1H-pyrazole with phosphoryl chloride in the presence of N,N-dimethylformamide, to obtain 1-methyl-1H-pyrazole-4-carbaldehyde;
treating 1-methyl-1H-pyrazole-4-carbaldehyde with hydroxylamine hydrochloride in the presence of ethanol, to obtain 1-methyl-1H-pyrazole-4-carbaldehyde oxime;
treating 1-methyl-1H-pyrazole-4-carbaldehyde oxime with sodium azide in the presence of ammonium chloride and N,N-dimethylformamide, to form 5-(1-methyl-1H-pyrazol-4-yl)-2H-tetrazole;
reacting 5-(1-methyl-1H-pyrazole-4-yl)-2H-tetrazole with methyl iodide or dimethyl sulphate, in the presence of potassium hydroxide or potassium carbonate to obtain 2-methyl-5-(1-methyl-1H-pyrazol-4-yl)-2H-tetrazole;
chlorinating 2-methyl-5-(1-methyl-1H-pyrazole-4-yl)-2H-tetrazole with N-chlorosuccinimide, in the presence acetonitrile, to obtain 5-(5-chloro-1-methyl-1H-pyrazol-4-yl)-2-methyl-2H-tetrazole;
treating 5-(5-chloro-1-methyl-1H-pyrazole-4-yl)-2-methyl-2H-tetrazole with benzyl thiol in the presence of a N,N-dimethylformamide and sodium hydride to obtain compound 5-(5-(benzylthio)-1-methyl-1H-pyrazol-4-yl)-2-methyl-2H-tetrazole of formula I, wherein R₁ is —SCH₂Ph;
treating 5-(5-(benzylthio)-1-methyl-1H-pyrazol-4-yl)-2-methyl-2H-tetrazole with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

22. The process as claimed in claim 1, comprising:
treating 1-methyl-1H-pyrazole with phosphoryl chloride in the presence of N,N-dimethylformamide, to obtain 1-methyl-1H-pyrazole-4-carbaldehyde;
treating 1-methyl-1H-pyrazole-4-carbaldehyde with hydroxylamine hydrochloride in the presence of ethanol, to obtain 1-methyl-1H-pyrazole-4-carbaldehyde oxime;
treating 1-methyl-1H-pyrazole-4-carbaldehyde oxime with sodium azide in the presence of ammonium chloride and N,N-dimethylformamide, to form the 5-(1-methyl-1H-pyrazol-4-yl)-2H-tetrazole;
reacting 5-(1-methyl-1H-pyrazole-4-yl)-2H-tetrazole with methyl iodide and dimethyl sulphate, in the presence of potassium hydroxide and potassium carbonate to obtain 2-methyl-5-(1-methyl-1H-pyrazol-4-yl)-2H-tetrazole;
chlorinating 2-methyl-5-(1-methyl-1H-pyrazole-4-yl)-2H-tetrazole with N-chlorosuccinimide, in the presence acetonitrile, to obtain 5-(5-chloro-1-methyl-1H-pyrazol-4-yl)-2-methyl-2H-tetrazole;
treating 5-(5-chloro-1-methyl-1H-pyrazole-4-yl)-2-methyl-2H-tetrazole with sodium hydrosulfide in the presence of a N,N-dimethylformamide to obtain the compounds (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof, of the formula 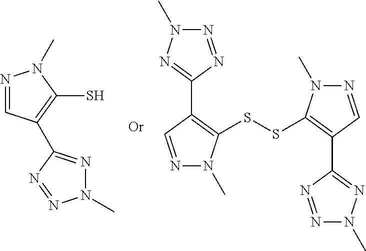
treating 1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof, with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

23. A process for preparation of azimsulfuron or its salts, isomers, and other derivatives thereof, comprising:
treating malononitrile with triethyl orthoformate in the presence of acetic anhydride to obtain 2-(ethoxymethylene) malononitrile;
treating 2-(ethoxymethylene) malononitrile with N-methyl hydrazine sulfate or N-methyl hydrazine and triethyl amine, in the presence of methanol to obtain 5-amino-1-methyl-1H-pyrazole-4-carbonitrile;
chlorinating 5-amino-1-methyl-1H-pyrazole-4-carbonitrile with sodium nitrite and cuprous chloride, with hydrochloric acid to obtain 5-chloro-1-methyl-1H-pyrazole-4-carbonitrile;
treating 5-chloro-1-methyl-1H-pyrazole-4-carbonitrile with sodium azide in the presence of either ammonium chloride, and N,N-dimethylformamide, or tri-n-butyl-tin chloride and toluene to obtain 5-(5-chloro-1-methyl-1H-pyrazole-4-yl)-2H-tetrazole);
methylating 5-(5-chloro-1-methyl-1H-pyrazole-4-yl)-2H-tetrazole with a methylating agent, such as dimethyl sulfate, methyl iodide or methyl bromide and potassium carbonate, in the presence of acetone to obtain 5-(5-chloro-1-methyl-1H-pyrazole-4-yl)-2-methyl-2H-tetrazole, or its regio-isomer 5-(5-chloro-1-methyl-1H-pyrazole-4-yl)-1-methyl-1H-tetrazole, or mixtures thereof;
treating 5-(5-chloro-1-methyl-1H-pyrazole-4-yl)-2-methyl-2H-tetrazole, or its regio-isomer 5-(5-chloro-1-methyl-1H-pyrazole-4-yl)-1-methyl-1H-tetrazole, or mixtures thereof with benzyl thiol in the presence of a N,N-dimethylformamide and sodium hydride to obtain compound 5-(5-(benzylthio)-1-methyl-1H-pyrazol-4-yl)-2-methyl-2H-tetrazole of formula I, wherein R₁ is —SCH₂Ph;
treating 5-(5-(benzylthio)-1-methyl-1H-pyrazol-4-yl)-2-methyl-2H-tetrazole with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

24. A process for preparation of azimsulfuron or its salts, isomers, and other derivatives thereof, comprising:
treating malononitrile with triethyl orthoformate in the presence of acetic anhydride to obtain 2-(ethoxymethylene) malononitrile;
treating 2-(ethoxymethylene) malononitrile with an N-methyl hydrazine sulfate and triethyl amine, in the presence of methanol to obtain 5-amino-1-methyl-1H-pyrazole-4-carbonitrile;
chlorinating the 5-amino-1-methyl-1H-pyrazole-4-carbonitrile with sodium nitrite and cuprous chloride, with hydrochloric acid to obtain 5-chloro-1-methyl-1H-pyrazole-4-carbonitrile;
treating 5-chloro-1-methyl-1H-pyrazole-4-carbonitrile with sodium azide in the presence of either ammonium chloride, and N,N-dimethylformamide, or tri-n-butyl tin chloride and toluene, to obtain 5-(5-chloro-1-methyl-1H-pyrazole-4-yl)-2H-tetrazole);
methylating 5-(5-chloro-1-methyl-1H-pyrazole-4-yl)-2H-tetrazole with dimethyl sulfate and potassium carbonate, in the presence of acetone to obtain 5-(5-chloro-1-methyl-1H-pyrazole-4-yl)-2-methyl-2H-tetrazole, or its regio-isomer 5-(5-chloro-1-methyl-1H-pyrazole-4-yl)-1-methyl-1H-tetrazole, or mixtures thereof;
treating 5-(5-chloro-1-methyl-1H-pyrazole-4-yl)-2-methyl-2H-tetrazole, or its regio-isomer 5-(5-chloro-1-methyl-1H-pyrazole-4-yl)-1-methyl-1H-tetrazole, or mixtures thereof with sodium hydrosulfide in the presence of a N,N-dimethylformamide to obtain the compounds (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof, of the formula 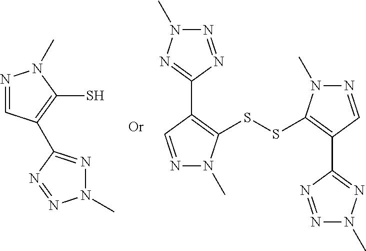
treating 1-methyl-4-(2-methyl-2H-tetrazol-5-yl)-1H-pyrazole-5-thiol, or its disulfide (1,2-bis-(1-methyl-4-(2-methyl-2H-tetrazol-5-yl)-1H-pyrazol-5-yl)disulfane), or mixture thereof, with aqueous acetic acid or formic acid and chlorine gas or sodium hypochlorite in presence of
hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

25. The process as claimed in claim 1, comprising:
treating 1-methyl-1H-pyrazole with iodine and hydrogen peroxide in aqueous medium to obtain 4-iodo-1-methyl-1H-pyrazole;
treating 4-iodo-1-methyl-1H-pyrazole with potassium ferrocyanide and palladium (II) acetate in the presence of sodium carbonate, and N,N-dimethylacetamide, to obtain 1-methyl-1H-pyrazole-4-carbonitrile;
treating 1-methyl-1H-pyrazole-4-carbonitrile with sodium azide and either with tri-n-butyl tin chloride in the presence of toluene, or ammonium chloride in the presence of N,N-dimethylformamide to obtain 5-(1-methyl-1H-pyrazol-4-yl)-2H-tetrazole;
reacting 5-(1-methyl-1H-pyrazol-4-yl)-2H-tetrazole with methyl iodide or dimethyl sulphate, in the presence of potassium hydroxide or potassium carbonate to obtain 2-methyl-5-(1-methyl-1H-pyrazol-4-yl)-2H-tetrazole;
chlorinating 2-methyl-5-(1-methyl-1H-pyrazol-4-yl)-2H-tetrazole with N-chlorosuccinimide, in the presence acetonitrile, to obtain 5-(5-chloro-1-methyl-1H-pyrazol-4-yl)-2-methyl-2H-tetrazole;
treating 5-(5-chloro-1-methyl-1H-pyrazole-4-yl)-2-methyl-2H-tetrazole with benzyl thiol in the presence of N,N-dimethylformamide and sodium hydride to obtain compound 5-(5-(benzylthio)-1-methyl-1H-pyrazol-4-yl)-2-methyl-2H-tetrazole of formula I, wherein R₁ is —SCH₂Ph;
treating 5-(5-(benzylthio)-1-methyl-1H-pyrazol-4-yl)-2-methyl-2H-tetrazole with aqueous acetic acid or formic acid and chlorine gas or sodium hypochlorite in presence of hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

26. The process as claimed in claim 1, comprising:
treating 1-methyl-1H-pyrazole with iodine and hydrogen peroxide in aqueous medium to obtain 4-iodo-1-methyl-1H-pyrazole;
treating 4-iodo-1-methyl-1H-pyrazole with potassium ferrocyanide and palladium (II) acetate in the presence of sodium carbonate, and N,N-dimethylacetamide, to obtain 1-methyl-1H-pyrazole-4-carbonitrile;
treating 1-methyl-1H-pyrazole-4-carbonitrile with sodium azide and either with tri-n-butyl tin chloride in the presence of toluene, or ammonium chloride in the presence of N,N-dimethylformamide to obtain 5-(1-methyl-1H-pyrazol-4-yl)-2H-tetrazole;
reacting 5-(1-methyl-1H-pyrazol-4-yl)-2H-tetrazole with methyl iodide and dimethyl sulphate, in the presence of potassium hydroxide and potassium carbonate to obtain 2-methyl-5-(1-methyl-1H-pyrazol-4-yl)-2H-tetrazole;
chlorinating 2-methyl-5-(1-methyl-1H-pyrazol-4-yl)-2H-tetrazole with N-chlorosuccinimide, in the presence acetonitrile, to obtain 5-(5-chloro-1-methyl-1H-pyrazol-4-yl)-2-methyl-2H-tetrazole;
treating 5-(5-chloro-1-methyl-1H-pyrazole-4-yl)-2-methyl-2H-tetrazole with sodium hydrosulfide in the presence of a N,N-dimethylformamide to obtain the compounds (1-methyl-4-(2-methyl-2H-tetrazol-5-yl)-1H-pyrazole-5-thiol), or its disulfide (1,2-bis-(1-methyl-4-(2-methyl-2H-tetrazol-5-yl)-1H-pyrazol-5-yl)disulfane), or mixture thereof, of the formula 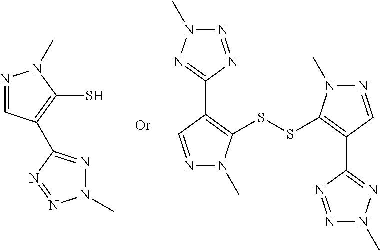
treating 1-methyl-4-(2-methyl-2H-tetrazol-5-yl)-1H-pyrazole-5-thiol, or its disulfide (1,2-bis-(1-methyl-4-(2-methyl-2H-tetrazol-5-yl)-1H-pyrazol-5-yl)disulfane), or mixture thereof, with aqueous acetic acid or formic acid and chlorine gas or sodium hypochlorite in presence of
hydrochloric acid in chlorinated solvents such as dichloromethane, 1,2-dichloroethane or with aqueous acetic acid and N-chlorosuccinimide or hydrogen peroxide in presence of hydrochloric acid in aqueous cyclic ether such as tetrahydrofuran, 1,4-dioxane to obtain 1-methyl-4-(2-methyl-2H-tetrazol-5-yl)-1H-pyrazole-5-sulfonyl chloride;
converting the 1-methyl-4-(2-methyl-2H-tetrazol-5-yl)-1H-pyrazole-5-sulfonyl chloride to 1-methyl-4-(2-methyl-2H-tetrazol-5-yl)-1H-pyrazole-5-sulfonamide in the presence of aqueous ammonia and 1,2-dichloroethane; and
treating the 1-methyl-4-(2-methyl-2H-tetrazol-5-yl)-1H-pyrazole-5-sulfonamide with phenyl (4,6-dimethoxypyrimidin-2-yl)carbamate in the presence of triethylamine and acetonitrile to obtain azimsulfuron or its salts, isomers, and other derivatives thereof.

27. The process as claimed in claim 6, wherein the alkali metal hydrosulfide is selected from sodium hydrosulfide or potassium hydrosulfide.

28. The process as claimed in claim 8, wherein the thiol is selected from C₁₋₄ alkyl thiol or benzyl thiol.
